(19) United States
(12) Patent Application Publication
(10) Pub. No.: US 2006/0240424 A1
(43) Pub. Date: Oct. 26, 2006

(54) Regulation of human p2y15 g protein-coupled receptor

(76) Inventors: Hisayo Inbe, Hyogo-ken (JP); Jeffrey Encinas, San Diego, CA (US)

Correspondence Address: JONES DAY, 222 EAST 41ST ST, NEW YORK, NY, 10017, US

(21) Appl. No.: 10/529,278

(22) PCT Filed: Sep. 25, 2003
(86) PCT No.: PCT/EP03/10666

§ 371 Date: Feb. 2, 2006

(30) Foreign Application Priority Data
Sep. 27, 2002 (US) 60413840
Jan. 28, 2003 (US) 60442891

(51) Int. Cl. C12Q 1/68; G01N 33/567; C12N 15/09

(52) U.S. Cl. 435/6; 435/7.2; 435/455

(57) ABSTRACT

Reagents which regulate human P2Y15 G protein-coupled receptor can play a role in preventing, ameliorating, or correcting bronchoconstriction or inflammation in diseases such as allergies including but not limited to asthma. Further, such reagents is useful to treat diseases of kidney function or disease related to mast cell.

### TECHNICAL FIELD OF THE INVENTION

The invention relates to the area of G protein-coupled receptors. More particularly, it relates to the area of P2Y15 G protein-coupled receptors and their regulation. It further relates to the treatment of bronchoconstriction and inflammation.

### BACKGROUND OF THE INVENTION

G Protein-Coupled Receptors

Many medically significant biological processes are mediated by signal transduction pathways that involve G proteins (Lefkowitz, Nature 351, 353-354, 1991). The family of G protein-coupled receptors (GPCR) includes receptors for hormones, neurotransmitters, growth factors, and viruses. Specific examples of GPCRs include receptors for such diverse agents as dopamine, calcitonin, adrenergic hormones, endothelin, cAMP, adenosine, acetylcholine, serotonin, histamine, thrombin, kinin, follicle stimulating hormone, opsins, endothelial differentiation gene-1, rhodopsins, odorants, cytomegalovirus, G proteins themselves, effector proteins such as phospholipase C, adenyl cyclase, and phosphodiesterase, and actuator proteins such as protein kinase A and protein kinase C.

GPCRs possess seven conserved membrane-spanning domains connecting at least eight divergent hydrophilic loops. GPCRs (also known as 7TM receptors) have been characterized as including these seven conserved hydrophobic stretches of about 20 to 30 amino acids, connecting at least eight divergent hydrophilic loops. Most GPCRs have single conserved cysteine residues in each of the first two extracellular loops, which form disulfide bonds that are believed to stabilize functional protein structure. The seven transmembrane regions are designated as TM1, TM2, TM3, TM4, TM5, TM6, and TM7. TM3 has been implicated in signal transduction. Phosphorylation and lipidation (palmitylation or farnesylation) of cysteine residues can influence signal transduction of some GPCRs. Most GPCRs contain potential phosphorylation sites within the third cytoplasmic loop and/or the carboxy terminus. For several GPCRs, such as the β-adrenergic receptor, phosphorylation by protein kinase A and/or specific receptor kinases mediates receptor desensitization.

For some receptors, the ligand binding sites of GPCRs are believed to comprise hydrophilic sockets formed by several GPCR transmembrane domains. The hydrophilic sockets are surrounded by hydrophobic residues of the GPCRs. The hydrophilic side of each GPCR transmembrane helix is postulated to face inward and form a polar ligand binding site. TM3 has been implicated in several GPCRs as having a ligand binding site, such as the TM3 aspartate residue. TM5 serines, a TM6 asparagine, and TM6 or TM7 phenylalanines or tyrosines also are implicated in ligand binding.

GPCRs are coupled inside the cell by heterotrimeric G-proteins to various intracellular enzymes, ion channels, and transporters (see Johnson et al., Endoc. Rev. 10, 317-331, 1989). Different G-protein alpha-subunits preferentially stimulate particular effectors to modulate various biological functions in a cell. Phosphorylation of cytoplasmic residues of GPCRs is an important mechanism for the regulation of some GPCRs. For example, in one form of signal transduction, the effect of hormone binding is the activation inside the cell of the enzyme, adenylate cyclase. Enzyme activation by hormones is dependent on the presence of the nucleotide GTP. GTP also influences hormone binding. A G protein connects the hormone receptor to adenylate cyclase. G protein exchanges GTP for bound GDP when activated by a hormone receptor. The GTP-carrying form then binds to activated adenylate cyclase. Hydrolysis of GTP to GDP, catalyzed by the G protein itself, returns the G protein to its basal, inactive form. Thus, the G protein serves a dual role, as an intermediate that relays the signal from receptor to effector, and as a clock that controls the duration of the signal.

Over the past 15 years, nearly 350 therapeutic agents targeting GPCRs have been successfully introduced onto the market. This indicates that these receptors have an established, proven history as therapeutic targets. Clearly, there is an on-going need for identification and characterization of further GPCRs which can play a role in preventing, ameliorating, or correcting dysfunctions or diseases including, but not limited to, infections such as bacterial, fungal, protozoan, and viral infections, particularly those caused by HIV viruses, pain, cancers, anorexia, bulimia, asthma, Parkinson's diseases, acute heart failure, hypotension, hypertension, urinary retention, osteoporosis, angina pectoris, myocardial infarction, ulcers, asthma, allergies, multiple sclerosis, benign prostatic hypertrophy, and psychotic and neurological disorders, including anxiety, schizophrenia, manic depression, delirium, dementia, several mental retardation, and dyskinesias, such as Huntington's disease and Tourett's syndrome.

P2Y Receptors

Extracellular nucleotides induce a wide variety of responses in many cell types, including muscle contraction and relaxation, vasodilation, neurotransmission, platelet aggregation, ion transport regulation, and cell growth. The effects are exerted mainly through two types of receptors: P2Y type G protein-coupled receptors, and P2X type ligand-gated ion channels. Nine distinct nucleotide-stimulated G protein-coupled receptors members have been characterized to date in humans. The nine receptors can be further subdivided into three groups according to ligand specificity: those activated by adenine nucleotides (P2Y1, P2Y11, P2Y12, and P2Y13), those activated by uridine nucleotides (P2Y4, P2Y6, CYSLT1, and GPR105), and those activated by both adenine and uridine nucleotides (P2Y2). The naturally occuring nucleotides that have been found to bind to these receptors have invariably been nucleotide diphosphates and nucleotide triphosphates. For example, ATP is the energy source for many biochemical reactions, a precursor for ribonucleic acid (RNA) synthesis, the precursor for cyclic AMP synthesis, etc. However, ATP also functions as an extracellular messenger in neuronal and non-neuronal tissues. Extracellular ATP exerts its effects on these tissues by acting through membrane-associated purinoreceptors (Burnstock, G. Ann. NY Acad. Sci. (1990) 603:1-17) which can be either ligand-gated ion channels (Bean, B. P. (1992) Trends Pharmac. Sci. 12:87-90; Bean, B. P. and Fried, D. D. (1990) Ion Channels 2:169-203) that are generally referred to as P2X receptors, (but also known as: purinergic channels, P2X R-channels, and ATP-gated channels) or G-protein-coupled (P2Y) receptors (Barnard, E. A. et al. (1994) Trends Pharmac. Sci. 15:67-70). See U.S. Pat. No. 5,856,129.

Adenosine Receptors

In addition to nucleotides, nucleosides have also been shown to have extracellular signaling functions. Adenosine, a purine nucleoside, is a ubiquitous modulator of numerous physiological activities, particularly within the cardiovascular and nervous systems. The effects of adenosine appear to be mediated by specific cell surface receptor proteins. Adenosine modulates diverse physiological functions including induction of sedation, vasodilation, suppression of cardiac rate and contractility, inhibition of platelet aggregability, stimulation of gluconeogenesis and inhibition of lipolysis. In addition to its effects on adenylate cyclase, adenosine has been shown to open potassium channels, reduce flux through calcium channels, and inhibit or stimulate phosphoinositide turnover through receptor-mediated mechanisms (See for example, C. E. Muller and B. Stein “Adenosine Receptor Antagonists: Structures and Potential Therapeutic Applications,” Current Pharmaceutical Design, 2:501 (1996) and C. E. Muller “A.sub.1-Adenosine Receptor Antagonists,” Exp. Opin. Ther. Patents 7(5):419 (1997)).

Adenosine receptors belong to the superfamily of purine receptors which are currently subdivided into P.sub.1 (adenosine) and P.sub.2 (ATP, AMP OR ADENOSINE RECEPTOR LIGAND, and other nucleotides) receptors. Four receptor subtypes for the nucleoside adenosine have been cloned so far from various species including humans. Two receptor subtypes (A.sub.1 and A.sub.2a) exhibit affinity for adenosine in the nanomolar range while two other known subtypes A.sub.2b and A.sub.3 are low-affinity receptors, with affinity for adenosine in the low-micromolar range. A.sub.1 and A.sub.3 adenosine receptor activation can lead to an inhibition of adenylate cyclase activity, while A.sub.2a and A.sub.2b activation causes a stimulation of adenylate cyclase.

Adenosine 5′-monophosphate (AMP) and Adenosine

All cells contain adenosine and adenine nucleotides, and many cell types have been shown to release adenosine or adenine nucleotides upon stimulation. For example, mast cells have been reported to release adenosine upon antigen challenge [Marquardt, D. L et al. (1984) Proc. Natl. Acad. Sci. U.S.A. 81:6192-6]. Activation of platelets [Jarvis, G. E. et al. (1996) Eur J Pharmacol 315:203-12], neutrophils [Madara, J. L. et al (1993) J. Clin. Invest. 91:2320-5)] and eosinophils [Resnick, M. B. et al. (1993)] has been reported to induce the release of AMP. Once released, adenine nucleotides also can be converted to adenosine by ectonucleotidase enzymes. Adenosine has been found to be increased in the bronchoalveolar lavage fluid (BALF) of inflamed airways and in particular is known to be present at higher concentrations in the BALF of patients with chronic inflammatory conditions of the lung, such as asthma and COPD (Driver, A. G. et al. (1993) Am. J. Respir. Dis. 148:91-7).

AMP or adenosine introduced artificially, either by inhalation or by instillation, into the respiratory tracts of patients with asthma and other inflammatory diseases of the lungs can cause immediate bronchoconstriction [Polosa, R. and Holgate, S. T. (1997) Thorax 52:919-23]. The response is significantly higher in patients with asthma than in other lung diseases, and is rarely seen in normal volunteers. Therefore it has recently been suggested that the response to AMP or adenosine can be used to differentiate asthma from other related diseases and that it can further be used as a specific marker of disease activity since responsiveness correlates well with inflammatory status. In light of the bronchoconstrictive activity of AMP and adenosine, it is clear that endogenously produced AMP and adenosine potentially play a significant role in the pathology of asthma and other inflammatory lung diseases. Indeed, two drugs that have bronchodilator effects in asthma, theophylline and enprofylline, are thought to achieve their effects by blocking the adenosine-induced activation of inflammatory cells and the subsequent release of inflammatory mediators.

P2Y15

WO0214511 discloses human P2Y15 receptor, its amino acid sequence and nucleotide sequence as well as their regulation. The mouse ortholog of P2Y15 is in the accession number XP_{—}139267.

Asthma

Asthma is thought to arise as a result of interactions between multiple genetic and environmental factors and is characterized by three major features: 1) intermittent and reversible airway obstruction caused by bronchoconstriction, increased mucus production, and thickening of the walls of the airways that leads to a narrowing of the airways, 2) airway hyperresponsiveness caused by a decreased control of airway caliber, and 3) airway inflammation. Certain cells are critical to the inflammatory reaction of asthma and they include T cells and antigen presenting cells, B cells that produce IgE, and mast cells, basophils, eosinophils, and other cells that bind IgE. These effector cells accumulate at the site of allergic reaction in the airways and release toxic products that contribute to the acute pathology and eventually to the tissue destruction related to the disorder. Other resident cells, such as smooth muscle cells, lung epithelial cells, mucus-producing cells, and nerve cells may also be abnormal in individuals with asthma and may contribute to the pathology. While the airway obstruction of asthma, presenting clinically as an intermittent wheeze and shortness of breath, is generally the most pressing symptom of the disease requiring immediate treatment, the inflammation and tissue destruction associated with the disease can lead to irreversible changes that eventually make asthma a chronic disabling disorder requiring long-term management.

Despite recent important advances in our understanding of the pathophysiology of asthma, the disease appears to be increasing in prevalence and severity (Gergen and Weiss, Am. Rev. Respir. Dis. 146, 823-24, 1992). It is estimated that 30-40% of the population suffer with atopic allergy, and 15% of children and 5% of adults in the population suffer from asthma (Gergen and Weiss, 1992). Thus, an enormous burden is placed on our health care resources. However, both diagnosis and treatment of asthma are difficult. The severity of lung tissue inflammation is not easy to measure and the symptoms of the disease are often indistinguishable from those of respiratory infections, chronic respiratory inflammatory disorders, allergic rhinitis, or other respiratory disorders. Often, the inciting allergen cannot be determined, making removal of the causative environmental agent difficult. Current pharmacological treatments suffer their own set of disadvantages. Commonly used therapeutic agents, such as beta agonists, can act as symptom relievers to transiently improve pulmonary function, but do not affect the underlying inflammation. Agents that can reduce the underlying inflammation, such as anti-inflammatory steroids, can have major drawbacks that range from immunosuppression to bone loss (Goodman and Gilman's THE PHARMACOLOGIC BASIS OF THERAPEUTICS, Seventh Edition, MacMillan Publishing Company, NY, USA, 1985). In addition, many of the present therapies, such as inhaled corticosteroids, are short-lasting, inconvenient to use, and must be used often on a regular basis, in some cases for life, making failure of patients to comply with the treatment a major problem and thereby reducing their effectiveness as a treatment.

Because of the problems associated with conventional therapies, alternative treatment strategies have been evaluated. Glycophorin A (Chu and Sharom, Cell. Immunol. 145, 223-39, 1992), cyclosporin (Alexander et al., Lancet 339, 324-28, 1992), and a nonapeptide fragment of IL-2 (Zav'yalov et al., Immunol. Lett. 31, 285-88, 1992) all inhibit interleukin-2 dependent T lymphocyte proliferation; however, they are known to have many other effects. For example, cyclosporin is used as a immuno-suppressant after organ transplantation. While these agents may represent alternatives to steroids in the treatment of asthmatics, they inhibit interleukin-2 dependent T lymphocyte proliferation and potentially critical immune functions associated with homeostasis. Other treatments that block the release or activity of mediators of bronchochonstriction, such as cromones or anti-leukotrienes, have recently been introduced for the treatment of mild asthma, but they are expensive and not effective in all patients and it is unclear whether they have any effect on the chronic changes associated with asthmatic inflammation. There remains a need in the art for the identification of a treatment that can act in pathways critical to the development of asthma that blocks both the episodic attacks of the disorder and preferentially dampens the hyperactive allergic immune response without immunocompromising the patient.

### SUMMARY OF THE INVENTION

The present invention is based on the discovery that G protein coupled receptor GPR80 (Lee, D K et al., 2001, Gene 275:83-91) is regulated in vivo by AMP and adenosine. It is referred to as P2Y15 G protein-coupled receptor hereinafter.

It is an object of the invention to provide reagents and methods for regulating a human P2Y15 G protein-coupled receptor. This and other objects of the invention are provided by one or more of the embodiments described below.

One embodiment of the invention is a method for detecting the activity of P2Y15 in a sample, which method comprises the steps of: a) incubating a sample with P2Y15 and a ligand under conditions which allow binding of P2Y15 and the ligand, and b) detecting a second messenger, wherein said ligand is AMP or adenosine receptor ligand.

The method may further comprises the steps of: a) incubating a second sample with P2Y15 in the absence of the ligand under conditions which allow binding of P2Y15 and the ligand, and b) detecting a second messenger. The sample may comprise cells expressing P2Y15 or cell membranes bearing P2Y15.

Another embodiment of the invention is a method of screening for an agent that modulate P2Y15 activity using cells expressing P2Y15. The method comprises a) incubating a first sample of said cells in the presence of said agent and a second sample of said cells in the absence of said agent, both said samples under conditions which allow binding of AMP or adenosine receptor ligand to P2Y15; b) detecting a signalling activity of P2Y15 polypeptide in said first and second samples, and c) comparing the results of said second messenger assays for said first and second samples.

Yet another embodiment of the invention is a method of screening for an agent to modulate P2Y15 activity using cell membranes bearing P2Y15. The method comprises a) incubating a first sample of said cell membranes in the presence of said agent and a second sample of said cell membranes in the absence of said agent, both said samples under conditions which allow binding of AMP or adenosine receptor ligand to P2Y15; b) detecting a signalling activity of P2Y15 polypeptide in said first and second samples, and c) comparing the results of said second messenger assays for said first and second samples. Further embodiment of the invention is a method for determining if a test compound increases or decreases the activity of P2Y15 using cells expressing P2Y15. The method comprises a) incubating a first sample of said cells in the presence of said test compound and a second sample of said cells in the absence of said test compound, both said samples under conditions which permit binding of AMP or adenosine receptor ligand to P2Y15; b) detecting a signalling activity of P2Y15 polypeptide in said first and second samples, and c) comparing the results of said second messenger assays for said first and second samples.

Another embodiment of the invention is a method for determining if a test compound increases or decreases the activity of P2Y15 using cell membranes bearing P2Y15. The method comprises: a) incubating a first sample of said cell membranes in the presence of said test compound and a second sample of said cell membranes in the absence of said test compound, both said samples under conditions which permit binding of AMP or adenosine receptor ligand to P2Y15; b) detecting a signalling activity of P2Y15 polypeptide in said first and second samples, and c) comparing the results of said second messenger assays for said first and second samples.

Another embodiment of the invention is a method of identifying an agent that modulates the function of P2Y15. The method comprises: a) contacting a P2Y15 polypeptide in the presence and absence of an agent under conditions permitting the binding of said AMP or adenosine receptor ligand to said P2Y15 polypeptide; and b) measuring the binding of said P2Y15 polypeptide to said agent, relative to the binding in the absence of said agent. The agent which changes binding is identified as a potential therapeutic agent for decreasing or increasing the function of P2Y15. The measuring is performed using a method selected from label displacement, surface plasmon resonance, fluorescence resonance energy transfer, fluorescence quenching, and fluorescence polarization. The agent may be selected from the group consisting of a natural or synthetic peptide, a polypeptide, an antibody or antigen-binding fragment thereof, a lipid, a carbohydrate, a nucleic acid, and a small organic molecule. The step of measuring a signalling activity of the P2Y15 polypeptide comprises detecting a change in the level of a second messenger.

The step of detecting a signalling activity may comprise measurement of guanine nucleotide binding or exchange, adenylate cyclase activity, cAMP, protein kinase C activity, phosphatidylinosotol breakdown, diacylglycerol, inositol triphosphate, intracellular calcium, arachinoid acid concentration, MAP kinase activity, tyrosine kinase activity, and reporter gene expression.

Further embodiment of the invention is a reagent that modulates the activity of a P2Y15 polypeptide or polynucleotide. The reagent is identified by any of the above method.

Another embodiment of the invention is a pharmaceutical composition, which comprises the above mentioned reagent and a pharmaceutically acceptable carrier.

### BRIEF DESCRIPTION OF THE DRAWINGS

FIG. 1 shows the relative expression of human P2Y15 GPCR in various human tissues and cells.

FIG. 2 shows calcium mobilization (A) and cyclic AMP generation (B) stimulated by AMP (filled symbols) and adenosine (empty symbols) in transfected HEK293 cells expressing P2Y15 (circles).

FIG. 3 shows specific binding of increasing concentrations of ³H-adenosine (A) and ³²P-AMP (B) to transfected HEK293 cells expressing P2Y15 (circles) and nontransfected HEK293 cells (squares). The binding of ³H-AMP (C) or ³H-adenosine (D) to P2Y15-transfected cells and nontransfected cells could be competed in both cases by unlabled AMP (filled symbols) or adenosine (open symbols).

FIG. 4 shows non-specific antagonists of adenosine block AMP- and adenosine-induced P2Y15 signaling. Calcium mobilization induced by 10 M AMP (A) or 10 M adenosine (B) was effectively blocked in a dose-dependent manner by theophylline, IBMX, 8-phenyltheophylline, caffeine, and AMP-CP. Kᵢ values for each of the antagonists are shown.

FIG. 5 shows the relative intensity of expression of P2Y15 and various well known genes and markers expressed in mast cells as measured by DNA microarray analysis.

### DETAILED DESCRIPTION OF THE INVENTION

Relevance of P2Y15 GPCR for the Treatment of Asthma

P2Y15 GPCR is a seven-transmembrane-domain molecule that has highest homology to P2Y1. It was originally found in a search for P2Y homologs in genomic sequence databases. Only one EST has been reported to date for this gene, from a cDNA library derived from normal human epithelium. The applicants performed expression profiling of this gene showing that it is expressed highest in the trachea, salivary glands, and kidneys, and less so in fetal brain, colon, placenta, and lung.

Although P2Y15 GPCR is closest in homology to P2Y1, which binds adenine nucleotides (ATP and AMP OR ADENOSINE RECEPTOR LIGAND), it also has significant homology to P2Y2 and P2Y4, which bind both A and U nucleotides, to P2Y3, which binds U nucleotides, and to leukotriene receptors, which bind LTB₄, LTC₄, and LTD₄. The applicants therefore deteremined the true ligand of this receptor empirically.

In studies of airway epithelia, both ATP and UTP have been found to equipotently regulate epithelial electrolyte and water transport, trigger mucin secretion, and increase ciliary beat frequency. In the trachea, nucleotides can induce tracheal gland serous cells, which are responsible for the secretion of antibacterial and anti-proteolytic proteins, to produce secretory leukocyte proteinase inhibitor and to increase chloride transport. Studies in a mouse knockout of the P2Y2 receptor show that it is the dominant extracellular nucleotide receptor in airway epithelium, but that other nucleotide receptors exist that function similarly in the respiratory tract.

Expression profiling studies of P2Y15 GPCR performed by the applicants show that it is expressed highly in tissues of the upper respiratory tract. Its high expression in the salivary glands and trachea indicate that it plays a role in exocrine secretion, which in the airways has mainly a protective role. In asthma, however, over-production of mucin contributes to the viscid mucus plugs that occlude asthmatic airways. Submucosal glands in the large airways of asthmatics also frequently show evidence of hyperplasia, which may somehow be due to overstimulation by external mediators.

Both agonists and antagonists of the P2Y15 receptor can have a beneficial effect in asthmatics. Agonists can increase protective protein secretion and increase ciliary beat rate, while antagonists can slow mucus production and glandular hyperplasia, prevent smooth muscle contraction, and reduce the sensitivity to respiratory tract irritants, including allergens, polluted, dry, or cold air, low oxygen concentrations, high CO₂ or CO concentrations, superoxides, and inflammatory mediators.

It has been discovered by the present applicant that the P2Y15 GPCR can be regulated to treat bronchoconstriction and inflammation in diseases such as allergies including but not limited to asthma. Human P2Y15 GPCR has the amino acid sequence shown in SEQ ID NO:2.

Human P2Y15 GPCR also can be used to screen for human P2Y15 GPCR agonists and antagonists.

Relevance of P2Y15 GPCR for the Treatment of Other Diseases

Within the kidney, analysis of P2Y15 transcript localization by in situ hybridization performed by the applicants showed that expression appears to be highest in the kidney microvasculature. Since theophylline and caffeine, which act as inhibitors of P2Y15 signaling, are known to have diuretic effects on the kidneys, other antagonists or agonists of P2Y15 are also expected to have effects on kidney function. Such antagonists and agonists of P2Y15 can be used to therapeutically regulate urine production, and in doing so, can also be beneficial in the regulation of the concentration of blood components, such as electrolytes and proteins. For example, P2Y15 antagonists or agonists can be used in the treament of diseases such as congestive heart failure, high blood pressure, edema, cirrhosis of the liver, and the nephrotic syndrome.

In an analysis performed by the applicants using DNA microarrays to detect the expression levels of gene transcripts in human umbilical cord blood-derived mast cells, the expression of P2Y15 transcripts was found to be, in terms of expression intensity, among the top 1% of genes expressed in these cells. Because of this extremely high expression, P2Y15 is considered by the applicants to be an important signaling molecule for mast cells. Consequently, regulation of P2Y15 signaling can be used as a method to treat diseases and conditions in which mast cells are involved. Increased numbers of mast cells are found in many pathological conditions. For example, mast cell hyperplasia in the skin (mastocytosis) manifests with skin lesions and may present with symptoms of urticaria and flushing due to the chemical mediators released during mast cell degranulation. Children may develop single mastocytomas or the multiple cutaneous lesions of urticaria pigrnentosa. In adults, multiple organ involvement can occur (notably affecting bone, liver, spleen and lymph nodes) even without apparent skin lesions (systemic mastocytosis). Lesions of the bone may be localised or widespread; osteoclastic or osteoblastic. Increased mast cell numbers are also seen in some inflammatory bowel diseases (ulcerative colitis, Crohn's disease) and in parasitic infections. Cutaneous neurofibromas, benign and malignant breast lesions, and some soft tissue tumours also show high numbers of mast cells. Mast cell numbers are also found to be high in interstitial cystitis and in idiopathic reduced bladder storage (sensory urge incontinence). Even in disease conditions where mast cell numbers are not recognized to be abnormally high, regulation of P2Y15 signaling in mast cells can have a beneficial effect. For example, the degranulation of inflammatory cells has been reported to lead to the inactivation of muscarinic receptors on presynaptic nerve endings, interfering with the inhibitory function of the receptors and leading to an increased release of acetylcholine into the synapse. Such inactivation may lead to abnormal muscle contractions and instability, such as that seen in asthma and in overactive bladder. Preventing dysregulated degranulation of inflammatory cells, such as mast cells, by regulating P2Y15 signaling can therefore be one way of treating symptomatic muscle contractions and instability.

Expression profiling studies of P2Y15 GPCR performed by the applicants show that it is expressed highly in the salivary glands and trachea. This indicates that antagonists or agonists of P2Y15 can be used to treat upper respiratory and oral diseases or conditions in which there is an abnormal production of mucus or saliva, such as Sjoegren's syndrome, dry mouth, dental carries, post-nasal drip, and cough.

Definition

As used herein the term “P2Y15” refers to a polypeptide that is encoded by any polynucleotide selected from the group consisting of
a) a polynucleotide encoding a P2Y15 polypeptide comprising an amino acid sequence selected from the group constisting of:
   amino acid sequences which are at least about 50% identical to the amino acid sequence shown in SEQ ID NO:2, 4, or 6; and
   the amino acid sequence shown in SEQ ID NO:2, 4, or 6
b) a polynucleotide comprising the sequence of SEQ ID NO: 1, 3, or 5;
c) a polynucleotide which hybridizes under stringent conditions to a polynucleotide specified in (a) and (b) and encodes a P2Y15 polypeptide;
d) a polynucleotide the nucleic acid sequence of which deviates from the nucleic acid sequences specified in (a) to (c) due to the degeneration of the genetic code and encodes a P2Y15; and
e) a polynucleotide, which represents a fragment, derivative or allelic variation of a nucleic acid sequence specified in (a) to (d) and encodes a P2Y15 and maintain P2Y15 activity.

As used herein the term “Ligand” refers to a molecule which binds to a receptor in a manner that is similar or equivalent to AMP.

As used herein the term “Adenosine receptor ligand” refers to specific and non-specific agonists and antagonists of adenosine receptors, including, but not limited to adenosine, 2-chloroadenosine, N6-Cyclopentyladenosine (CPA), CGS-21680 hydro-chloride, Chloro-IB-MECA, 5′-(N-Ethylcarboxamido) adenosine (NECA) and its derivatives, 8-Phenyltheophylline (8-PT), 3-isobutyl-1 methylxanthine (IBMX), Alloxazine, 8-(p-Sulfophenyl) theophylline (8-SPT), 8-cyclopentyl-1,3-dipropyl-xanthine (DPCPX), caffeine, theophylline, and enprofylline. Other adenosine receptor ligands are also known to those skilled in the art, e.g., as disclosed in Pharmacological reviews 53:527-552, 2001.

As used herein the term “Second messenger” refers to a molecule, generated or caused to vary in concentration by the activation of a G-Protein Coupled Receptor, that participates in the transduction of a signal from that GPCR. Non-limiting examples of second messengers include cAMP, diacylglycerol, inositol triphosphate, arachidonic acid metabolites, calcium ions. The term “change in the level of a second messenger” refers to an increase or decrease of at least 10% in the detected level of a given second messenger relative to the amount detected in an assay performed in the absence of a candidate modulator.

As used herein the term “Second messenger assay” preferably comprises the measurement of guanine nucleotide binding or exchange, adenylate cyclase, intracellular cAMP, intracellular inositol phosphate, intracellular diacylglycerol concentration, arachidonic acid concentration, calcium mobilization, MAP kinase(s) or tyrosine kinase(s), protein kinase C activity, or reporter gene expression or an aequorin-based assay according to methods known in the art and defined herein.

As used herein the term “Sample” is the source of molecules being tested for the presence of an agent or modulator compound that modulates binding to or signalling activity of a P2Y15 polypeptide. A sample can be an environmental sample, a natural extract of animal, plant yeast or bacterial cells or tissues, a clinical sample, a synthetic sample, or a conditioned medium from recombinant cells or a fermentation process.

As used herein, the term “membrane fraction” refers to a preparation of cellular lipid membranes comprising a P2Y15 polypeptide. As the term is used herein, a “membrane fraction” is distinct from a cellular homogenate, in that at least a portion (i.e., at least 10%, and preferably more) of non-membrane-associated cellular constituents has been removed. The term “membrane associated” refers to those cellular constituents that are either integrated into a lipid membrane or are physically associated with a component that is integrated into a lipid membrane.

As used herein the term “Conditions which allow the binding of P2Y15 and a ligand” refers to conditions of, for example, temperature, salt concentration, pH and protein concentration under which a ligand binds P2Y15. Exact binding conditions will vary depending upon the nature of the assay, for example, whether the assay uses viable cells or only membrane fraction of cells.

Polypeptides

P2Y15 GPCR polypeptides according to the invention comprise at least 10, 12, 15, 20, 24, 30, 40, 50, 75, 100, 125, 150, 175, 200, 225, 250, 275, 300, 325, or 350 contiguous amino acids selected from the amino acid sequence shown in SEQ ID NO:2 or a biologically active variant of that sequence, as defined below. A P2Y15 GPCR polypeptide of the invention therefore can be a portion of a P2Y15 GPCR, a full-length P2Y15 GPCR, or a fusion protein comprising all or a portion of a P2Y15 GPCR.

A phylogenetic analysis comparing the protein sequence with other G protein-coupled receptors (GPCRs) places the molecule among a cluster of other P2Y receptors, distant from the known receptors for adenosine (van den Berge, M., Kerstjens, H. A., and Postna, D. S. (2002) Clin Exp Allergy 32, 824-830). The gene sequence is found on the genomic contig NT_{—}009952 which has been localized to human chromosome 13q32.

Biologically Active Variants

P2Y15 GPCR polypeptide variants which are biologically active, ie., retain the ability to bind a ligand to produce a biological effect, such as cyclic AMP formation, mobilization of intracellular calcium, or phosphoinositide metabolism, also are P2Y 15 GPCR polypeptides. Preferably, naturally or non-naturally occurring P2Y 15 GPCR polypeptide variants have amino acid sequences which are at least about 50, 55, 60, 65, 70, more preferably about 75, 90, 96, or 98% identical to an amino acid sequence shown in SEQ ID NO:2 or a fragment thereof. Percent identity between a putative P2Y15 GPCR polypeptide variant and an amino acid sequence of SEQ ID NO:2 is determined by conventional methods. See, for example, Altschul et al., Bull. Math. Bio. 48:603 (1986), and Henikoff & Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff & Henikoff, 1992.

Those skilled in the art appreciate that there are many established algorithms available to align two amino acid sequences. The “FASTA” similarity search algorithm of Pearson & Lipman is a suitable protein alignment method for examing the level of identity shared by an amino acid sequence disclosed herein and the amino acid sequence of a putative variant. The FASTA algorithm is described by Pearson & Lipman, Proc. Nat'l Acad. Sci. USA 85:2444(1988), and by Pearson, Meth. Enzymol. 183:63 (1990). Briefly, FASTA first characterizes sequence similarity by identifying regions shared by the query sequence (e.g., SEQ ID NO: 2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman & Wunsch, J. Mol. Biol. 48:444 (1970); Sellers, SIAM J. Appl. Math. 26:787 (1974)), which allows for amino acid insertions and deletions. Preferred parameters for FASTA analysis are: ktup=1, gap opening penalty-10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a P2Y15 GPCR polypeptide can be found using computer programs well known in the art, such as DNASTAR software. Whether an amino acid change results in a biologically active P2Y15 GPCR polypeptide can readily be determined by assaying for binding to a ligand or by conducting a functional assay, as described for example, in the specific Examples, below.

Fusion Proteins

Fusion proteins are useful for generating antibodies against P2Y15 GPCR polypeptide amino acid sequences and for use in various assay systems. For example, fusion proteins can be used to identify proteins which interact with portions of a P2Y15 GPCR polypeptide. Protein affinity chromatography or library-based assays for protein-protein interactions, such as the yeast two-hybrid or phage display systems, can be used for this purpose. Such methods are well known in the art and also can be used as drug screens.

A P2Y15 GPCR polypeptide fusion protein comprises two polypeptide segments fused together by means of a peptide bond. The first polypeptide segment comprises at least 10, 12, 15, 20, 24, 30, 40, 50, 75, 100, 125, 150, 175, 200, 225, 250, 275, 300, or 325 contiguous amino acids of SEQ ID NO:2 or a biologically active variant of SEQ ID NO:2. Contiguous amino acids for use in a fusion protein can be selected from the amino acid sequence shown in SEQ ID NO:2 or from a biologically active variant of those sequences, such as those described above. The first polypeptide segment also can comprise full-length P2Y15 G protein-coupled receptor.

The second polypeptide segment can be a full-length protein or a protein fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions.

A fusion protein also can be engineered to contain a cleavage site located between the P2Y15 GPCR polypeptide-encoding sequence and the heterologous protein sequence, so that the P2Y15 GPCR polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences of SEQ ID NO:1 in proper reading frame with nucleotides encoding the second polypeptide segment and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif.), Santa Cruz Bio-technology (Santa Cruz, Calif.), MBL International Corporation (MIC; Watertown, Mass.), and Quantum Biotechnologies (Montreal, Canada; 1-888-DNA-KITS).

Identification of Species Homologs

Species homologs of human P2Y15 GPCR polypeptide can be obtained using P2Y15 GPCR polynucleotides (described below) to make suitable probes or primers for screening cDNA expression libraries from other species, such as guinea pigs, monkeys, or yeast, identifying cDNAs which encode homologs of P2Y15 GPCR polypeptide, and expressing the cDNAs as is known in the art.

Species homologs of human P2Y15 GPCR polypeptide can also be obtained by performing a search, using the human or other species's P2Y15 polypeptide sequences as a query, in the Genbank database of the National Center for Bio-technology Information (http://www.ncbi.nlm.nih.gov) using the program tblastn. Mouse ortholog of P2Y15 can be found under accession number XP_{—}139267.

Polynucleotides

A P2Y15 GPCR polynucleotide can be single- or double-stranded and comprises a coding sequence or the complement of a coding sequence for a P2Y15 GPCR polypeptide. A nucleotide sequence encoding SEQ ID NO:2 is shown in SEQ ID NO: 1.

Degenerate nucleotide sequences encoding human P2Y15 GPCR polypeptides, as well as homologous nucleotide sequences which are at least about 50, 55, 60, 65, or 70, more preferably about 75, 90, 96, or 98% identical to a nucleotide sequence shown in SEQ ID NOS:1, 3, or 5 or its complement also are P2Y15 GPCR polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of P2Y15 GPCR polynucleotides which encode biologically active P2Y15 GPCR polypeptides also are P2Y15 GPCR polynucleotides.

Identification of Polynucleotide Variants and Homologs

Variants and homologs of the P2Y15 GPCR polynucleotides described above also are P2Y15 GPCR polynucleotides. Typically, homologous P2Y15 GPCR polynucleotide sequences can be identified by hybridization of candidate polynucleotides to known P2Y15 GPCR polynucleotides under stringent conditions, as is known in the art. For example, using the following wash conditions—2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each—homologous sequences can be identified which contain at most about 25-30% basepair mismatches. More preferably, homologous nucleic acid strands contain 15-25% basepair mismatches, even more preferably 5-15% basepair mismatches.

Species homologs of the P2Y15 GPCR polynucleotides disclosed herein also can be identified by making suitable probes or primers and screening cDNA expression libraries from other species, such as mice, monkeys, or yeast. Human variants of P2Y15 GPCR polynucleotides can be identified, for example, by screening human cDNA expression libraries. It is well known that the Tₘ of a double-stranded DNA decreases by 1-1.5° C. with every 1% decrease in homology (Bonner et al., J. Mol. Biol. 81, 123 (1973). Variants of human P2Y15 GPCR polynucleotides or P2Y15 GPCR polynucleotides of other species can therefore be identified by hybridizing a putative homologous P2Y15 GPCR polynucleotide with a polynucleotide having a nucleotide sequence of SEQ ID NO:1, 3 or 5 or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to P2Y15 GPCR polynucleotides or their complements following stringent hybridization and/or wash conditions also are P2Y15 GPCR polynucleotides. Stringent wash conditions are well known and understood in the art and are disclosed, for example, in Sambrook et al., MOLECULAR CLONING: A LABORATORY MANUAL, 2d ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a P2Y15 GPCR polynucleotide having a nucleotide sequence shown in SEQ ID NO:1, 3, or 5 or the complement thereof and a polynucleotide sequence which is at least about 50, 55, 60, 65, 70, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.−16.6(log₁₀[Na⁺])+0.41(% G+C)−0.63(% formamide)−600/l), where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Preparation of Polynucleotides

A P2Y15 GPCR polynucleotide can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated P2Y15 GPCR polynucleotides. For example, restriction enzymes and probes can be used to isolate polynucleotide fragments which comprises P2Y15 GPCR nucleotide sequences. Isolated polynucleotides are in preparations which are free or at least 70, 80, or 90% free of other molecules.

P2Y15 GPCR cDNA molecules can be made with standard molecular biology techniques, using P2Y15 GPCR mRNA as a template. P2Y15 GPCR cDNA molecules can thereafter be replicated using molecular biology techniques known in the art and disclosed in manuals such as Sambrook et al. (1989). An amplification technique, such as PCR, can be used to obtain additional copies of polynucleotides of the invention, using either human genomic DNA or cDNA as a template.

Alternatively, synthetic chemistry techniques can be used to synthesizes P2Y15 GPCR polynucleotides. The degeneracy of the genetic code allows alternate nucleotide sequences to be synthesized which will encode a P2Y15 GPCR polypeptide having, for example, the amino acid sequence shown in SEQ ID NO:2, 4, or 6 or a biologically active variant thereof.

Extending Polynucleotides

Various PCR-based methods can be used to extend the nucleic acid sequences encoding the disclosed portions of human P2Y15 GPCR polypeptide to detect upstream sequences such as promoters and regulatory elements. For example, restriction-site PCR uses universal primers to retrieve unknown sequence adjacent to a known locus (Sarkar, PCR Methods Applic. 2, 318-322, 1993). Genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region (Triglia et al., Nucleic Acids Res. 16, 8186, 1988). Primers can be designed using commercially available software, such as OLIGO 4.06 Primer Analysis software (National Biosciences Inc., Plymouth, Minn.), to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which can be used is capture PCR, which involves PCR amplification of DNA fragments adjacent to a known sequence in human and yeast artificial chromosome DNA (Lagerstrom et al., PCR Methods Applic. 1, 111-119, 1991). In this method, multiple restriction enzyme digestions and ligations also can be used to place an engineered double-stranded sequence into an unknown fragment of the DNA molecule before performing PCR.

Another method which can be used to retrieve unknown sequences is that of Parker et al., Nucleic Acids Res. 19, 3055-3060, 1991). Additionally, PCR, nested primers, and PROMOTERFINDER libraries (CLONTECH, Palo Alto, Calif.) can be used to walk genomic DNA (CLONTECH, Palo Alto, Calif.). This process avoids the need to screen libraries and is useful in finding intron/exon junctions.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ non-transcribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g. GENOTYPER and Sequence NAVIGATOR, Perkin Elmer), and the entire process from loading of samples to computer analysis and electronic data display can be computer controlled. Capillary electrophoresis is especially preferable for the sequencing of small pieces of DNA which might be present in limited amounts in a particular sample.

Obtaining Polypeptides

P2Y15 GPCR polypeptides can be obtained, for example, by purification from cells, by expression of P2Y15 GPCR polynucleotides, or by direct chemical synthesis.

Protein Purification

P2Y15 GPCR polypeptides can be purified from any cell which expresses the receptor, including host cells which have been transfected with P2Y15 GPCR polynucleotides which express such polypeptides. A purified P2Y15 GPCR polypeptide is separated from other compounds which normally associate with the P2Y15 GPCR polypeptide in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis.

A P2Y15 GPCR polypeptide can be conveniently isolated as a complex with its associated G protein, as described in the specific examples, below. A preparation of purified P2Y15 GPCR polypeptides is at least 80% pure; preferably, the preparations are 90%, 95%, or 99% pure. Purity of the preparations can be assessed by any means known in the art, such as SDS-polyacrylamide gel electrophoresis.

Expression of Polynucleotides

To express a P2Y15 GPCR polypeptide, a P2Y15 GPCR polynucleotide can be inserted into an expression vector which contains the necessary elements for the transcription and translation of the inserted coding sequence. Methods which are well known to those skilled in the art can be used to construct expression vectors containing sequences encoding P2Y15 GPCR polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS IN MOLECULAR BIOLOGY, John Wiley & Sons, New York, N.Y., 1989.

A variety of expression vector/host systems can be utilized to contain and express sequences encoding a P2Y15 GPCR polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those non-translated regions of the vector—enhancers, promoters, 5′ and 3′ untranslated regions—which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 pfasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding a P2Y15 GPCR polypeptide, vectors based on SV40 or EBV can be used with an appropriate selectable marker.

Bacterial and Yeast Expression Systems

In bacterial systems, a number of expression vectors can be selected depending upon the use intended for the P2Y15 GPCR polypeptide. For example, when a large quantity of a P2Y15 GPCR polypeptide is needed for the induction of antibodies, vectors which direct high level expression of fusion proteins that are readily purified can be used. Such vectors include, but are not limited to, multifunctional E. coli cloning and expression vectors such as BLUESCRIPT (Stratagene). In a BLUESCRIPT vector, a sequence encoding the P2Y15 GPCR polypeptide can be ligated into the vector in frame with sequences for the amino-terminal Met and the subsequent 7 residues of β-galactosidase so that a hybrid protein is produced. pIN vectors (Van Heeke & Schuster, J. Biol. Chem. 264, 5503-5509, 1989) or pGEX vectors (Promega, Madison, Wis.) also can be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

In the yeast Saccharomyces cerevisiae, a number of vectors containing constitutive or inducible promoters such as alpha factor, alcohol oxidase, and PGH can be used. For reviews, see Ausubel et al. (1989) and Grant et al., Methods Enzymol. 153, 516-544, 1987.

Plant and Insect Expression Systems

If plant expression vectors are used, the expression of sequences encoding P2Y15 GPCR polypeptides can be driven by any of a number of promoters. For example, viral promoters such as the 35S and 19S promoters of CaMV can be used alone or in combination with the omega leader sequence from TMV (Takamatsu, EMBO J. 6, 307-311, 1987). Alternatively, plant promoters such as the small subunit of RUBISCO or heat shock promoters can be used (Coruzzi et al., EMBO J. 3, 1671-1680, 1984; Broglie et al., Science 224, 838-843, 1984; Winter et al., Results Probl. Cell Differ. 17, 85-105, 1991). These constructs can be introduced into plant cells by direct DNA transformation or by pathogen-mediated transfection. Such techniques are described in a number of generally available reviews (e.g., Hobbs or Murray, in MCGRAW HILL YEARBOOK OF SCIENCE AND TECHNOLOGY, McGraw Hill, New York, N.Y., pp. 191-196, 1992).

An insect system also can be used to express a P2Y15 GPCR polypeptide. For example, in one such system Autographa californica nuclear polyhedrosis virus (AcNPV) is used as a vector to express foreign genes in Spodoptera frugiperda cells or in Trichoplusia larvae. Sequences encoding P2Y15 GPCR polypeptides can be cloned into a non-essential region of the virus, such as the polyhedrin gene, and placed under control of the polyhedrin promoter. Successful insertion of P2Y15 GPCR polypeptides will render the polyhedrin gene inactive and produce recombinant virus lacking coat protein. The recombinant viruses can then be used to infect S. frugiperda cells or Trichoplusia larvae in which P2Y15 GPCR polypeptides can be expressed (Engelhard et al., Proc. Nat. Acad. Sci. 91, 3224-3227, 1994).

Mammalian Expression Systems

A number of viral-based expression systems can be used to express P2Y15 GPCR polypeptides in mammalian host cells. For example, if an adenovirus is used as an expression vector, sequences encoding P2Y15 GPCR polypeptides can be ligated into an adenovirus transcription/translation complex comprising the late promoter and tripartite leader sequence. Insertion in a non-essential E1 or E3 region of the viral genome can be used to obtain a viable virus which is capable of expressing a P2Y15 GPCR polypeptide in infected host cells (Logan & Shenk, Proc. Natl. Acad. Sci. 81, 3655-3659, 1984). If desired, transcription enhancers, such as the Rous sarcoma virus (RSV) enhancer, can be used to increase expression in mammalian host cells.

Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles).

Specific initiation signals also can be used to achieve more efficient translation of sequences encoding P2Y15 GPCR polypeptides. Such signals include the ATG initiation codon and adjacent sequences. In cases where sequences encoding a P2Y15 GPCR polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals (including the ATG initiation codon) should be provided. The initiation codon should be in the correct reading frame to ensure translation of the entire insert. Exogenous translational elements and initiation codons can be of various origins, both natural and synthetic. The efficiency of expression can be enhanced by the inclusion of enhancers which are appropriate for the particular cell system which is used (see Scharf et al., Results Probl. Cell Differ. 20, 125-162, 1994).

Host Cells

A host cell strain can be chosen for its ability to modulate the expression of the inserted sequences or to process the expressed P2Y15 GPCR polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

Stable expression is preferred for long-term, high-yield production of recombinant proteins. For example, cell lines which stably express P2Y15 GPCR polypeptides can be transformed using expression vectors which can contain viral origins of replication and/or endogenous expression elements and a selectable marker gene on the same or on a separate vector. Following the introduction of the vector, cells can be allowed to grow for 1-2 days in an enriched medium before they are switched to a selective medium. The purpose of the selectable marker is to confer resistance to selection, and its presence allows growth and recovery of cells which successfully express the introduced P2Y15 GPCR sequences. Resistant clones of stably transformed cells can be proliferated using tissue culture techniques appropriate to the cell type. See, for example, ANIMAL CELL CULTURE, R. I. Freshney, ed., 1986.

Any number of selection systems can be used to recover transformed cell lines. These include, but are not limited to, the herpes simplex virus thymidine kinase (Wigler et al., Cell 11, 223-32, 1977) and adenine phosphoribosyltransferase (Lowy et al., Cell 22, 817-23, 1980) genes which can be employed in tk⁻ or aprt⁻ cells, respectively. Also, antimetabolite, antibiotic, or herbicide resistance can be used as the basis for selection. For example, dhfr confers resistance to methotrexate (Wigler et al., Proc. Natl. Acad. Sci. 77, 3567-70, 1980), npt confers resistance to the aminoglycosides, neomycin and G-418 (Colbere-Garapin et al., J. Mol. Biol. 150, 1-14, 1981), and als and pat confer resistance to chlorsulfuron and phosphinotricin acetyltransferase, respectively (Murray, 1992, supra). Additional selectable genes have been described. For example, trpB allows cells to utilize indole in place of tryptophan, or hisD, which allows cells to utilize histinol in place of histidine (Hartman & Mulligan, Proc. Natl. Acad. Sci. 85, 8047-51, 1988). Visible markers such as anthocyanins, β-glucuronidase and its substrate GUS, and luciferase and its substrate luciferin, can be used to identify transformants and to quantify the amount of transient or stable protein expression attributable to a specific vector system (Rhodes et al., Methods Mol. Biol. 55, 121-131, 1995).

Detecting Expression of Polypeptides

Although the presence of marker gene expression suggests that the P2Y15 GPCR polynucleotide is also present, its presence and expression may need to be confirmed. For example, if a sequence encoding a P2Y15 GPCR polypeptide is inserted within a marker gene sequence, transformed cells containing sequences which encode a P2Y15 GPCR polypeptide can be identified by the absence of marker gene function. Alternatively, a marker gene can be placed in tandem with a sequence encoding a P2Y15 GPCR polypeptide under the control of a single promoter. Expression of the marker gene in response to induction or selection usually indicates expression of the P2Y15 GPCR polynucleotide.

Alternatively, host cells which contain a P2Y15 GPCR polynucleotide and which express a P2Y15 GPCR polypeptide can be identified by a variety of procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a polynucleotide sequence encoding a P2Y15 GPCR polypeptide can be detected by DNA-DNA or DNA-RNA hybridization or amplification using probes or fragments or fragments of polynucleotides encoding a P2Y15 GPCR polypeptide. Nucleic acid amplification-based assays involve the use of oligonucleotides selected from sequences encoding a P2Y15 GPCR polypeptide to detect transformants which contain a P2Y15 GPCR polynucleotide.

A variety of protocols for detecting and measuring the expression of a P2Y15 GPCR polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a P2Y15 GPCR polypeptide can be used, or a competitive binding assay can be employed. These and other assays are described in Hampton et al., SEROLOGICAL METHODS: A LABORATORY MANUAL, APS Press, St. Paul, Minn., 1990) and Maddox et al., J. Exp. Med 158, 1211-1216, 1983).

A wide variety of labels and conjugation techniques are known by those skilled in the art and can be used in various nucleic acid and amino acid assays. Means for producing labeled hybridization or PCR probes for detecting sequences related to polynucleotides encoding P2Y15 GPCR polypeptides include oligolabeling, nick translation, end-labeling, or PCR amplification using a labeled nucleotide. Alternatively, sequences encoding a P2Y15 GPCR polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

Expression and Purification of Polypeptides

Host cells transformed with nucleotide sequences encoding a P2Y15 GPCR polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides which encode P2Y15 GPCR polypeptides can be designed to contain signal sequences which direct secretion of soluble P2Y15 GPCR polypeptides through a prokaryotic or eukaryotic cell membrane or which direct the membrane insertion of membrane-bound P2Y15 GPCR polypeptide.

As discussed above, other constructions can be used to join a sequence encoding a P2Y15 GPCR polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Inclusion of cleavable linker sequences such as those specific for Factor Xa or enterokinase (Invitrogen, San Diego, Calif.) between the purification domain and the P2Y15 GPCR polypeptide also can be used to facilitate purification. One such expression vector provides for expression of a fusion protein containing a P2Y15 GPCR polypeptide and 6 histidine residues preceding a thioredoxin or an enterokinase cleavage site. The histidine residues facilitate purification by IMAC (immobilized metal ion affinity chromatography, as described in Porath et al., Prot. Exp. Purif 3, 263-281, 1992), while the enterokinase cleavage site provides a means for purifying the P2Y15 GPCR polypeptide from the fusion protein. Vectors which contain fusion proteins are disclosed in Kroll et al., DNA Cell Biol. 12, 441-453, 1993.

Chemical Synthesis

Sequences encoding a P2Y15 GPCR polypeptide can be synthesized, in whole or in part, using chemical methods well known in the art (see Caruthers et al., Nucl. Acids Res. Symp. Ser. 215-223, 1980; Horn et al. Nucl. Acids Res. Symp. Ser. 225-232, 1980). Alternatively, a P2Y15 GPCR polypeptide itself can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perkin Elmer). Optionally, fragments of P2Y15 GPCR polypeptides can be separately synthesized and combined using chemical methods to produce a full-length molecule.

The newly synthesized peptide can be substantially purified by preparative high performance liquid chromatography (e.g., Creighton, PROTEINS: STRUCTURES AND MOLECULAR PRINCIPLES, WH Freeman and Co., New York, N.Y., 1983). The composition of a synthetic P2Y15 GPCR polypeptide can be confirmed by amino acid analysis or sequencing (e.g., the Edman degradation procedure; see Creighton, supra). Additionally, any portion of the amino acid sequence of the P2Y15 GPCR polypeptide can be altered during direct synthesis and/or combined using chemical methods with sequences from other proteins to produce a variant polypeptide or a fusion protein.

Production of Altered Polypeptides

As will be understood by those of skill in the art, it may be advantageous to produce P2Y15 GPCR polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art to alter P2Y15 GPCR polypeptide-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Orthologs

Orthologs, such as the mouse ortholog of P2Y15 GPCR (GenBank accession number XP_{—}139267.1; SEQ ID NO: 3/amino acid seq; SEQ ID NO:4), may also be produced. The mouse ortholog has an amino acid sequence that is 85% identical and 88% homologous to the human P2Y15 GPCR amino acid sequence. It will be appreciated by one of skill in the art that due to the high degree of identity between the mouse and the human proteins that the mouse can be used as a model system to screen for and study the effect of target compounds that effect P2Y15 GPCR activity. The mouse can also be used as a model system to study the effect of mutations in the P2Y15 gene or over- or under-expression of P2Y15 GPCR on disease, including but not limited to allergic responses. The mouse ortholog of P2Y15 GPCR is also useful as a purified protein to screen for target compounds that modulate receptor activity. This protein may also'be purified to aid in efforts to solve the structure of P2Y15 GPCR and the design of small molecules that effect protein activity. The rat ortholog of P2Y15 was found by applicant by using the mouse protein sequence in a tblastn query against the rat genome trace sequences subset of Genbank. Seven transmembrane regions as predicted by the computer program TMpred are indicated with heavy overlines and numbered TM1-7. The Genbank accession number of the rat P2Y15 sequence is AY191367 (SEQ ID NO:5/amino acid seq; SEQ ID NO:6).

Antibodies

Any type of antibody known in the art can be generated to bind specifically to an epitope of a P2Y15 GPCR polypeptide. “Antibody” as used herein includes intact immunoglobulin molecules, as well as fragments thereof, such as Fab, F(ab′)₂, and Fv, which are capable of binding an epitope of a P2Y15 GPCR polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of a P2Y15 GPCR polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a P2Y15 GPCR polypeptide provides a detection signal at least 5-, 10-, or 20-fold higher than a detection signal provided with other proteins when used in an immunochemical assay. Preferably, antibodies which specifically bind to P2Y15 GPCR polypeptides do not detect other proteins in immunochemical assays and can immunoprecipitate a P2Y15 GPCR polypeptide from solution.

P2Y15 GPCR polypeptides can be used to immunize a mammal, such as a mouse, rat, rabbit, guinea pig, monkey, or human, to produce polyclonal antibodies. If desired, a P2Y15 GPCR polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a P2Y15 GPCR polypeptide can be prepared using any technique which provides for the production of antibody molecules by continuous cell lines in culture. These techniques include, but are not limited to, the hybridoma technique, the human B-cell hybridoma technique, and the EBV-hybridoma technique (Kohler et al., Nature 256, 495-497, 1985; Kozbor et al., J. Immunol. Methods 81, 31-42, 1985; Cote et al., Proc. Natl. Acad. Sci. 80, 2026-2030, 1983; Cole et al., Mol. Cell Biol. 62, 109-120, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-6855, 1984; Neuberger et al., Nature 312, 604-608, 1984; Takeda et al., Nature 314, 452-454, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, humanized antibodies can be produced using recombinant methods, as described in GB2188638B. Antibodies which specifically bind to a P2Y15 GPCR polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to P2Y15 GPCR polypeptides. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., 1996, Eur. J. Cancer Prev. 5, 507-11). Single-chain antibodies can be mono- or bispecific, and can be bivalent or tetravalent. Construction of tetravalent, bispecific single-chain antibodies is taught, for example, in Coloma & Morrison, 1997, Nat. Biotechnol. 15, 159-63. Construction of bivalent, bispecific single-chain antibodies is taught in Mallender & Voss, 1994, J. Biol. Chem. 269, 199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., 1995, Int. J. Cancer 61, 497-501; Nicholls et al., 1993, J. Immunol. Meth. 165, 81-91).

Antibodies which specifically bind to P2Y15 GPCR polypeptides also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833-3837, 1989; Winter et al., Nature 349, 293-299, 1991).

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies according to the invention can be purified by methods well known in the art. For example, antibodies can be affinity purified by passage over a column to which a P2Y15 GPCR polypeptide is bound. The bound antibodies can then be eluted from the column using a buffer with a high salt concentration.

Antisense Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of P2Y15 GPCR gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20, 1-8, 1994; Sonveaux, Meth. Mol. Biol. 26, 1-72, 1994; Uhlmann et al., Chem. Rev. 90, 543-583, 1990.

Modifications of P2Y15 GPCR gene expression can be obtained by designing antisense oligonucleotides which will form duplexes to the control, 5′, or regulatory regions of the P2Y15 GPCR. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994). An antisense oligonucleotide also can be designed to block translation of mRNA by preventing the transcript from binding to ribosomes.

Precise complementarity is not required for successful complex formation between an antisense oligonucleotide and the complementary sequence of a P2Y15 GPCR polynucleotide. Antisense oligonucleotides which comprise, for example, 2, 3, 4, or 5 or more stretches of contiguous nucleotides which are precisely complementary to a P2Y15 GPCR polynucleotide, each separated by a stretch of contiguous nucleotides which are not complementary to adjacent P2Y15 GPCR nucleotides, can provide sufficient targeting specificity for P2Y15 GPCR mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular P2Y15 GPCR polynucleotide sequence.

Antisense oligonucleotides can be modified without affecting their ability to hybridize to a P2Y15 GPCR polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., Trends Biotechnol. 10, 152-158, 1992; Uhlmann et al., Chem. Rev. 90, 543-584, 1990; Uhlmann et al., Tetrahedron. Lett. 215, 3539-3542, 1987.

Ribozymes

Ribozymes are RNA molecules with catalytic activity. See, e.g., Cech, Science 236, 1532-1539; 1987; Cech, Ann. Rev. Biochem. 59, 543-568; 1990, Cech, Curr. Opin. Struct. Biol. 2, 605-609; 1992, Couture & Stinchcomb, Trends Genet. 12, 510-515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of a P2Y15 GPCR polynucleotide can be used to generate ribozymes which will specifically bind to mRNA transcribed from the P2Y15 GPCR polynucleotide. Methods of designing and constructing ribozymes which can cleave other RNA molecules in trans in a highly sequence specific manner have been developed and described in the art (see Haseloff et al. Nature 334, 585-591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201).

Specific ribozyme cleavage sites within a P2Y15 GPCR RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate P2Y15 GPCR RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease P2Y15 GPCR expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, ribozymes can be engineered so that ribozyme expression will occur in response to factors which induce expression of a target gene. Ribozymes also can be engineered to provide an additional level of regulation, so that destruction of mRNA occurs only when both a ribozyme and a target gene are induced in the cells.

Differentially Expressed Genes

Described herein are methods for the identification of genes whose products interact with human P2Y15 G protein-coupled receptor. Such genes may represent genes that are differentially expressed in disorders including, but not limited to, CNS disorders, cardiovascular disorders, asthma, osteoporosis, diabetes, and COPD. Further, such genes may represent genes that are differentially regulated in response to manipulations relevant to the progression or treatment of such diseases. Additionally, such genes may have a temporally modulated expression, increased or decreased at different stages of tissue or organism development. A differentially expressed gene may also have its expression modulated under control versus experimental conditions. In addition, the human P2Y15 G protein-coupled receptor gene or gene product may itself be tested for differential expression.

The degree to which expression differs in a normal versus a diseased state need only be large enough to be visualized via standard characterization techniques such as differential display techniques. Other such standard characterization techniques by which expression differences may be visualized include but are not limited to, quantitative RT (reverse transcriptase), PCR, and Northern analysis.

Identification of Differentially Expressed Genes

To identify differentially expressed genes total RNA or, preferably, mRNA is isolated from tissues of interest. For example, RNA samples are obtained from tissues of experimental subjects and from corresponding tissues of control subjects. Any RNA isolation technique that does not select against the isolation of mRNA may be utilized for the purification of such RNA samples. See, for example, Ausubel et al., ed., CURRENT PROTOCOLS IN MOLECULAR BIOLOGY, John Wiley & Sons, Inc. New York, 1987-1993. Large numbers of tissue samples may readily be processed using techniques well known to those of skill in the art, such as, for example, the single-step RNA isolation process of Chomczynski, U.S. Pat. No. 4,843,155.

Transcripts within the collected RNA samples that represent RNA produced by differentially expressed genes are identified by methods well known to those of skill in the art. They include, for example, differential screening (Tedder et al., Proc. Natl. Acad. Sci. U.S.A. 85, 208-12, 1988), subtractive hybridization (Hedrick et al., Nature 308, 149-53; Lee et al., Proc. Natl. Acad. Sci. U.S.A. 88, 2825, 1984), and differential display (Liang & Pardee, Science 257, 967-71, 1992; U.S. Pat. No. 5,262,311), and microarrays.

The differential expression information may itself suggest relevant methods for the treatment of disorders involving the human P2Y15 G protein-coupled receptor. For example, treatment may include a modulation of expression of the differentially expressed genes and/or the gene encoding the human P2Y15 G protein-coupled receptor. The differential expression information may indicate whether the expression or activity of the differentially expressed gene or gene product or the human P2Y15 G protein-coupled receptor gene or gene product are up-regulated or down-regulated.

Screening Methods

The invention provides assays for screening test compounds which bind to or modulate the activity of a P2Y15 GPCR polypeptide or a P2Y15 GPCR polynucleotide. A test compound preferably binds to a P2Y15 GPCR polypeptide or polynucleotide. More preferably, a test compound decreases or increases a biological effect mediated via human P2Y15 GPCR by at least about 10, preferably about 50, more preferably about 75, 90, or 100% relative to the absence of the test compound.

Test Compounds

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. USA. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409).

High Throughput Screening

Test compounds can be screened for the ability to bind to P2Y15 GPCR polypeptides or polynucleotides or to affect P2Y15 GPCR activity or P2Y15 GPCR gene expression using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format.

Alternatively, “free format assays,” or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. U.S.A. 19, 1614-18 (1994). The cells are placed under agarose in petri dishes, then beads that carry combinatorial compounds are placed on the surface of the agarose. The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, “Strategies for Screening Combinatorial Libraries: Novel and Traditional Approaches,” reported at the First Annual Conference of The Society for Biomolecular Screening in Philadelphia, Pa. (Nov. 7-10, 1995). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV-light. Compounds that inhibited the enzyme were observed as local zones of inhibition having less color change.

Yet another example is described by Salmon et al., Molecular Diversity 2, 57-63 (1996). In this example, combinatorial libraries were screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Binding Assays

For binding assays, the test compound is preferably a small molecule which binds to and occupies the active site of the P2Y15 GPCR polypeptide, thereby making the ligand binding site inaccessible to substrate such that normal biological activity is prevented. Examples of such small molecules include, but are not limited to, small peptides or peptide-like molecules. In addition to adenosine and adenosine monophosphate, other potential ligands which may bind to a polypeptide of the invention include, but are not limited to, the natural ligands of known GPCRs and analogues or derivatives thereof. Natural ligands of GPCRs include adrenomedullin, amylin, calcitonin gene related protein (CGRP), calcitonin, anandamide, serotonin, histamine, adrenalin, noradrenalin, platelet activating factor, thrombin, C5a, bradykinin, and chemokines. Other potential ligands which may bind to a polypeptide of the invention include, but are not limited to, specific and non-specific agonists and antagonists of Adenosine receptors, including caffeine, theophylline, enprofylline, and IBMX.

In binding assays, either the test compound or the P2Y15 GPCR polypeptide can comprise a detectable label, such as a fluorescent, radioisotopic, chemiluminescent, or enzymatic label, such as horseradish peroxidase, alkaline phosphatase, or luciferase. Detection of a test compound which is bound to the P2Y15 GPCR polypeptide can then be accomplished, for example, by direct counting of radio-emmission, by scintillation counting, or by determining conversion of an appropriate substrate to a detectable product.

Alternatively, binding of a test compound to a P2Y15 GPCR polypeptide can be determined without labeling either of the interactants. For example, a micro-physiometer can be used to detect binding of a test compound with a P2Y15 GPCR polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and a P2Y15 GPCR polypeptide (McConnell et al., Science 257, 1906-1912, 1992).

Determining the ability of a test compound to bind to a P2Y15 GPCR polypeptide also can be accomplished using a technology such as real-time Bimolecular Interaction Analysis (BIA) (Sjolander & Urbaniczky, Anal. Chem. 63, 2338-2345, 1991, and Szabo et al., Curr. Opin. Struct. Biol. 5, 699-705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, a P2Y15 GPCR polypeptide can be used as a “bait protein” in a two-hybrid assay or three-hybrid assay (see, e.g., U.S. Pat. No. 5,283,317; Zervos et al., Cell 72, 223-232, 1993; Madura et al., J. Biol. Chem. 268, 12046-12054, 1993; Bartel et al., BioTechniques 14, 920-924, 1993; Iwabuchi et al., Oncogene 8, 1693-1696, 1993; and Brent WO94/10300), to identify other proteins which bind to or interact with the P2Y15 GPCR polypeptide and modulate its activity.

The two-hybrid system is based on the modular nature of most transcription factors, which consist of separable DNA-binding and activation domains. Briefly, the assay utilizes two different DNA constructs. For example, in one construct, polynucleotide encoding a P2Y15 GPCR polypeptide can be fused to a polynucleotide encoding the DNA binding domain of a known transcription factor (e.g., GAL-4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein which interacts with the P2Y15 GPCR polypeptide.

It may be desirable to immobilize either the P2Y15 GPCR polypeptide (or polynucleotide) or the test compound to facilitate separation of bound from unbound forms of one or both of the interactants, as well as to accommodate automation of the assay. Thus, either the P2Y15 GPCR polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the P2Y15 GPCR polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to a P2Y15 GPCR polypeptide (or polynucleotide) can be accomplished in any vessel suitable for containing the reactants. Examples of such vessels include microtiter plates, test tubes, and microcentrifuge tubes.

In one embodiment, the P2Y15 GPCR polypeptide is a fusion protein comprising a domain that allows the P2Y15 GPCR polypeptide to be bound to a solid support. For example, glutathione-S-transferase fusion proteins can be adsorbed onto glutathione sepharose beads (Sigma Chemical) or glutathione derivatized microtiter plates, which are then combined with the test compound or the test compound and the non-adsorbed P2Y15 GPCR polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either a P2Y15 GPCR polypeptide (or polynucleotide) or a test compound can be immobilized utilizing conjugation of biotin and streptavidin. Biotinylated P2Y15 GPCR polypeptides (or polynucleotides) or test compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to a P2Y15 GPCR polypeptide, polynucleotide, or a test compound, but which do not interfere with a desired binding site, such as the active site of the P2Y15 GPCR polypeptide, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the P2Y15 GPCR polypeptide or test compound, enzyme-linked assays which rely on detecting an activity of the P2Y 15 GPCR polypeptide, and SDS gel electrophoresis under non-reducing conditions.

Screening for test compounds which bind to a P2Y15 GPCR polypeptide or polynucleotide also can be carried out in an intact cell. Any cell which comprises a P2Y15 GPCR polypeptide or polynucleotide can be used in a cell-based assay system. A P2Y15 GPCR polynucleotide can be naturally occurring in the cell or can be introduced using techniques such as those described above. Binding of the test compound to a P2Y15 GPCR polypeptide or polynucleotide is determined as described above.

Functional Assays

Test compounds can be tested for the ability to increase or decrease a biological effect of a P2Y15 GPCR polypeptide. Such biological effects can be determined using the functional assays described in the specific examples, below. Functional assays can be carried out after contacting either a purified P2Y15 GPCR polypeptide, a cell membrane preparation, or an intact cell with a test compound. A test compound which decreases a functional activity of a P2Y15 GPCR by at least about 10, preferably about 50, more preferably about 75, 90, or 100% is identified as a potential agent for decreasing P2Y15 GPCR activity. A test compound which increases P2Y15 GPCR activity by at least about 10, preferably about 50, more preferably about 75, 90, or 100% is identified as a potential agent for increasing P2Y15 GPCR activity.

One such screening procedure involves the use of melanophores which are transfected to express a P2Y15 GPCR polypeptide. Such a screening technique is described in WO 92/01810 published Feb. 6, 1992. Thus, for example, such an assay may be employed for screening for a compound which inhibits activation of the receptor polypeptide by contacting the melanophore cells which comprise the receptor with both a receptor ligand and a test compound to be screened. Inhibition of the signal generated by the ligand indicates that a test compound is a potential antagonist for the receptor, i.e., inhibits activation of the receptor. The screen may be employed for identifying a test compound which activates the receptor by contacting such cells with compounds to be screened and determining whether each test compound generates a signal, i.e., activates the receptor.

Other screening techniques include the use of cells which express a human P2Y15 GPCR polypeptide (for example, transfected CHO cells) in a system which measures extracellular pH changes caused by receptor activation (see, e.g., Science 246, 181-296, 1989). For example, test compounds may be contacted with a cell which expresses a human P2Y15 GPCR polypeptide and a second messenger response, e.g., signal transduction or pH changes, can be measured to determine whether the test compound activates or inhibits the receptor.

Another such screening technique involves introducing RNA encoding a human P2Y15 GPCR polypeptide into Xenopus oocytes to transiently express the receptor. The transfected oocytes can then be contacted with the receptor ligand and a test compound to be screened, followed by detection of inhibition or activation of a calcium signal in the case of screening for test compounds which are thought to inhibit activation of the receptor.

Another screening technique involves expressing a human P2Y15 GPCR polypeptide in cells in which the receptor is linked to a phospholipase C or D. Such cells include endothelial cells, smooth muscle cells, embryonic kidney cells, etc. The screening may be accomplished as described above by quantifying the degree of activation of the receptor from changes in the phospholipase activity.

Details of functional assays such as those described above are provided in the specific examples, below.

Gene Expression

In another embodiment, test compounds which increase or decrease P2Y15 GPCR gene expression are identified. A P2Y15 GPCR polynucleotide is contacted with a test compound, and the expression of an RNA or polypeptide product of the P2Y15 GPCR polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of P2Y15 GPCR mRNA or polypeptide expression in the cells can be determined by methods well known in, the art for detecting mRNA or polypeptide. Either qualitative or quantitative methods can be used. The presence of polypeptide products of a P2Y15 GPCR polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into a P2Y15 GPCR polypeptide.

Such screening can be carried out either in a cell-free assay system or in an intact cell. Any cell which expresses a P2Y15 GPCR polynucleotide can be used in a cell-based assay system. The P2Y15 GPCR polynucleotide can be naturally occurring in the cell or can be introduced using techniques such as those described above. Either a primary culture or an established cell line, such as CHO or human embryonic kidney 293 cells, can be used.

Pharmaceutical Compositions

The invention also provides pharmaceutical compositions which can be administered to a patient to achieve a therapeutic effect. Pharmaceutical compositions of the invention can comprise, for example, a P2Y15 GPCR polypeptide, P2Y15 GPCR polynucleotide, antibodies which specifically bind to a P2Y15 GPCR polypeptide, or mimetics, agonists, antagonists, or inhibitors of a P2Y15 GPCR polypeptide activity. The compositions can be administered alone or in combination with at least one other agent, such as stabilizing compound, which can be administered in any sterile, bio-compatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically-acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrapulmonary, intrahepatic, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxy-propylmethyl-cellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Further details on techniques for formulation and administration can be found in the latest edition of REMINGTON'S PHARMACEUTICAL SCIENCES (Maack Publishing Co., Easton, Pa.). After pharmaceutical compositions have been prepared, they can be placed in an appropriate container and labeled for treatment of an indicated condition. Such labeling would include amount, frequency, and method of administration.

Therapeutic Indications and Methods

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense nucleic acid molecule, a specific antibody, ribozyme, or a P2Y15 GPCR polypeptide binding molecule) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

A reagent which affects P2Y15 GPCR activity can be administered to a human cell, either in vitro or in vivo, to reduce P2Y15 GPCR activity. The reagent preferably binds to an expression product of a human P2Y15 GPCR gene. If the expression product is a protein, the reagent is preferably an antibody. For treatment of human cells ex vivo, an antibody can be added to a preparation of stem cells which have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 10⁶ cells, more preferably about 1.0 μg of DNA per 16 nmole of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell types, such as a cell-specific ligand exposed on the outer surface of the liposome.

Complexing a liposome with a reagent such as an antisense oligonucleotide or ribozyme can be achieved using methods which are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al. Trends in Biotechnol. 11, 202-05 (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24 (1988); Wu et al., J. Biol. Chem. 269, 542-46 (1994); Zenke et al., Proc. Natl. Acad. Sci. U.S.A. 87, 3655-59 (1990); Wu et al., J. Biol. Chem. 266, 338-42 (1991).

Determination of a Therapeutically Effective Dose

The determination of a therapeutically effective dose is well within the capability of those skilled in the art. A therapeutically effective dose refers to that amount of active ingredient which increases or decreases P2Y15 GPCR activity relative to the P2Y15 GPCR activity which occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Effective in vivo dosages of an antibody are in the range of about 5 μg to about 50 μg/kg, about 50 μg to about 5 mg/kg, about 100 μg to about 500 μg/kg of patient body weight, and about 200 to about 250 μg/kg of patient body weight. For administration of polynucleotides encoding single-chain antibodies, effective in vivo dosages are in the range of about 100 ng to about 200 ng, 500 ng to about 50 mg, about 1 μg to about 2 mg, about 5 μg to about 500 μg, and about 20 μg to about 100 μg of DNA.

If the expression product is mRNA, the reagent is preferably an antisense oligonucleotide or a ribozyme. Polynucleotides which express antisense oligonucleotides or ribozymes can be introduced into cells by a variety of methods, as described above.

Preferably, a reagent reduces expression of a P2Y15 GPCR gene or the activity of a P2Y15 GPCR polypeptide by at least about 10, preferably about 50, more preferably about 75, 90, or 100% relative to the absence of the reagent. The effectiveness of the mechanism chosen to decrease the level of expression of a P2Y15 GPCR gene or the activity of a P2Y 15 GPCR polypeptide can be assessed using methods well known in the art, such as hybridization of nucleotide probes to P2Y15 GPCR-specific mRNA, quantitative RT-PCR, immunologic detection of a P2Y15 GPCR polypeptide, or measurement of P2Y15 GPCR activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, mammals such as dogs, cats, cows, horses, rabbits, monkeys, and most preferably, humans.

Diagnostic Methods

GPCRs also can be used in diagnostic assays for detecting diseases and abnormalities or susceptibility to diseases and abnormalities related to the presence of mutations in the nucleic acid sequences which encode a GPCR. Such diseases, by way of example, are related to cell transformation, such as tumors and cancers, and various cardiovascular disorders, including hypertension and hypotension, as well as diseases arising from abnormal blood flow, abnormal angiotensin-induced aldosterone secretion, and other abnormal control of fluid and electrolyte homeostasis.

According to the present invention, differences can be determined between the cDNA or genomic sequence encoding a P2Y15 GPCR in individuals afflicted with a disease and in normal individuals. If a mutation is observed in some or all of the afflicted individuals but not in normal individuals, then the mutation is likely to be the causative agent of the disease.

Sequence differences between a reference gene and a gene having mutations can be revealed by the direct DNA sequencing method. In addition, cloned DNA segments can be employed as probes to detect specific DNA segments. The sensitivity of this method is greatly enhanced when combined with PCR. For example, a sequencing primer can be used with a double-stranded PCR product or a single-stranded template molecule generated by a modified PCR. The sequence determination is performed by conventional procedures using radiolabeled nucleotides or by automatic sequencing procedures using fluorescent tags.

Genetic testing based on DNA sequence differences can be carried out by detection of alteration in electrophoretic mobility of DNA fragments in gels with or without denaturing agents. Small sequence deletions and insertions can be visualized, for example, by high resolution gel electrophoresis. DNA fragments of different sequences can be distinguished on denaturing formamide gradient gels in which the mobilities of different DNA fragments are retarded in the gel at different positions according to their specific melting or partial melting temperatures (see, e.g., Myers et al., Science 230, 1242, 1985). Sequence changes at specific locations can also be revealed by nuclease protection assays, such as RNase and S 1 protection or the chemical cleavage method (e.g., Cotton et al., Proc. Natl. Acad. Sci. USA 85, 4397-4401, 1985). Thus, the detection of a specific DNA sequence can be performed by methods such as hybridization, RNase protection, chemical cleavage, direct DNA sequencing or the use of restriction enzymes and Southern blotting of genomic DNA. In addition to direct methods such as gel-electrophoresis and DNA sequencing, mutations can also be detected by in situ analysis.

Altered levels of a P2Y15 GPCR also can be detected in various tissues. Assays used to detect levels of the receptor polypeptides in a body sample, such as blood or a tissue biopsy, derived from a host are well known to those of skill in the art and include radioimmunoassays, competitive binding assays, Western blot analysis, and ELISA assays.

All patents and patent applications cited in this disclosure are expressly incorporated herein by reference. The above disclosure generally describes the present invention. A more complete understanding can be obtained by reference to the following specific examples which are provided for purposes of illustration only and are not intended to limit the scope of the invention.

### EXAMPLE 1

Detection of P2Y15 GPCR Activity

The polynucleotide of SEQ ID NO: 1, 3, or 5 is inserted into the expression vector pCEV4 and the expression vector pCEV4-P2Y15 GPCR polypeptide obtained is transfected into human embryonic kidney 293 cells. These cells are scraped from a culture flask into 5 ml of Tris HCl, 5 mM EDTA, pH 7.5, and lysed by sonication. Cell lysates are centrifuged at 1000 rpm for 5 minutes at 4° C. The supernatant is centrifuged at 30,000×g for 20 minutes at 4° C. The pellet is suspended in binding buffer containing 50 mM Tris HCl, 5 mM MgSO₄, 1 mM EDTA, 100 mM NaCl, pH 7.5, supplemented with 0.1% BSA, 2 μg/ml aprotinin, 0.5 mg/ml leupeptin, and 10 μg/ml phosphoramidon. Optimal membrane suspension dilutions, defined as the protein concentration required to bind less than 10% of the added radioligand, are added to 96-well polypropylene microtiter plates containing ¹²⁵I-labeled ligand or test compound, non-labeled peptides, and binding buffer to a final volume of 250 μl.

In equilibrium saturation binding assays, membrane preparations are incubated in the presence of increasing concentrations (0.1 nM to 4 nM) of ¹²⁵I-labeled ligand or test compound (specific activity 2200 Ci/mmol). The binding affinities of different test compounds are determined in equilibrium competition binding assays, using 0.1 nM ¹²⁵I-peptide in the presence of twelve different concentrations of each test compound.

Binding reaction mixtures are incubated for one hour at 30° C. The reaction is stopped by filtration through GF/B filters treated with 0.5% polyethyleneimine, using a cell harvester. Radioactivity is measured by scintillation counting, and data are analyzed by a computerized non-linear regression program.

Non-specific binding is defined as the amount of radioactivity remaining after incubation of membrane protein in the presence of 100 nM of unlabeled peptide. Protein concentration is measured by the Bradford method using Bio-Rad Reagent, with bovine serum albumin as a standard. It is shown that the polypeptide of SEQ ID NO: 2 has a P2Y15 GPCR activity.

### EXAMPLE 2

Expression of Recombinant Human P2Y15 GPCR

The Pichia pastoris expression vector pPICZB (Invitrogen, San Diego, Calif.) is used to produce large quantities of a human P2Y15′ GPCR polypeptides in yeast. The human P2Y15 GPCR polypeptide-encoding DNA sequence is derived from the nucleotide sequence shown in SEQ ID NO:1. Before insertion into vector pPICZB the DNA sequence is modified by well known methods in such a way that it contains at its 5′-end an initiation codon and at its 3′-end an enterokinase cleavage site, a His6 reporter tag and a termination codon. Moreover, at both termini recognition sequences for restriction endonucleases are added and after digestion of the multiple cloning site of pPICZ B with the corresponding restriction enzymes the modified polypeptide encoding DNA sequence is ligated into pPICZB. This expression vector is designed for inducible expression in Pichia pastoris, expression is driven by a yeast promoter. The resulting pPICZ/md-His6 vector is used to transform the yeast.

The yeast are cultivated under usual conditions in 5 liter shake flasks and the recombinantly produced protein isolated from the culture by affinity chromatography (Ni-NTA-Resin) in the presence of 8 M urea. The bound polypeptide is eluted with buffer, pH 3.5, and neutralized. Separation of the P2Y15 GPCR polypeptide from the His6 reporter tag is accomplished by site-specific proteolysis using enterokinase (Invitrogen, San Diego, Calif.) according to manufacturer's instructions. Purified human P2Y15 GPCR polypeptide is obtained.

### EXAMPLE 3

Radioligand Binding Assays

Human embryonic kidney 293 cells transfected with a polynucleotide which expresses human P2Y15 GPCR are scraped from a culture flask into 5 ml of Tris HCl, 5 mM EDTA, pH 7.5, and lysed by sonication. Cell lysates are centrifuged at 1000 rpm for 5 minutes at 4° C. The supernatant is centrifuged at 30,000×g for 20 minutes at 4° C. The pellet is suspended in binding buffer containing 50 mM Tris HCl, 5 mM MgSO₄, 1 mM EDTA, 100 mM NaCl, pH 7.5, supplemented with 0.1% BSA, 2 μg/ml aprotinin, 0.5 mg/ml leupeptin, and 10 μg/ml phosphoramidon. Optimal membrane suspension dilutions, defined as the protein concentration required to bind less than 10% of the added radioligand, are added to 96-well polypropylene microtiter plates containing ¹²⁵I-labeled ligand or test compound, non-labeled peptides, and binding buffer to a final volume of 250 μl.

In equilibrium saturation binding assays, membrane preparations are incubated in the presence of increasing concentrations (0.1 nM to 4 nM) of ¹²⁵I-labeled ligand or test compound (specific activity 2200 Ci/mmol). The binding affinities of different test compounds are determined in equilibrium competition binding assays, using 0.1 nM ¹²⁵I-peptide in the presence of twelve different concentrations of each test compound.

Binding reaction mixtures are incubated for one hour at 30° C. The reaction is stopped by filtration through GF/B filters treated with 0.5% polyethyleneimine, using a cell harvester. Radioactivity is measured by scintillation counting, and data are analyzed by a computerized non-linear regression program.

Non-specific binding is defined as the amount of radioactivity remaining after incubation of membrane protein in the presence of 100 nM of unlabeled peptide. Protein concentration is measured by the Bradford method using Bio-Rad Reagent, with bovine serum albumin as a standard. A test compound which increases the radioactivity of membrane protein by at least 15% relative to radioactivity of membrane protein which was not incubated with a test compound is identified as a compound which binds to a human P2Y15 GPCR polypeptide.

### EXAMPLE 4

Effect of a Test Compound on Human P2Y15 GPCR-Mediated Cyclic AMP Formation

Receptor-mediated inhibition of cAMP formation can be assayed in host cells which express human P2Y15 GPCR. Cells are plated in 96-well plates and incubated in Dulbecco's phosphate buffered saline (PBS) supplemented with 10 mM HEPES, 5 mM theophylline, 2 μg/ml aprotinin, 0.5 mg/ml leupeptin, and 10 μg/ml phosphoramidon for 20 minutes at 37° C. in 5% CO₂. A test compound is added and incubated for an additional 10 minutes at 37° C. The medium is aspirated, and the reaction is stopped by the addition of 100 mM HCl. The plates are stored at 4° C. for 15 minutes. cAMP content in the stopping solution is measured by radioimmunoassay.

Radioactivity is quantified using a gamma counter equipped with data reduction software. A test compound which decreases radioactivity of the contents of a well relative to radioactivity of the contents of a well in the absence of the test compound is identified as a potential inhibitor of cAMP formation. A test compound which increases radioactivity of the contents of a well relative to radioactivity of the contents of a well in the absence of the test compound is identified as a potential enhancer of cAMP formation.

### EXAMPLE 5

Effect of a Test Compound on the Mobilization of Intracellular Calcium

Intracellular free calcium concentration can be measured by microspectrofluorometry using the fluorescent indicator dye Fura-2/AM (Bush et al., J. Neurochem. 57, 562-74, 1991). Stably transfected cells are seeded onto a 35 mm culture dish containing a glass coverslip insert. Cells are washed with HBS, incubated with a test compound, and loaded with 100 μl of Fura-2/AM (10 μM) for 20-40 minutes. After washing with HBS to remove the Fura-2/AM solution, cells are equilibrated in HBS for 10-20 minutes. Cells are then visualized under the 40× objective of a Leitz Fluovert FS microscope.

Fluorescence emission is determined at 510 nM, with excitation wavelengths alternating between 340 nM and 380 nM. Raw fluorescence data are converted to calcium concentrations using standard calcium concentration curves and software analysis techniques. A test compound which increases the fluorescence by at least 15% relative to fluorescence in the absence of a test compound is identified as a compound which mobilizes intracellular calcium.

### EXAMPLE 6

Effect of a Test Compound on Phosphoinositide Metabolism

Cells which stably express human P2Y15 GPCR cDNA are plated in 96-well plates and grown to confluence. The day before the assay, the growth medium is changed to 100 μl of medium containing 1% serum and 0.5 μCi ³H-myinositol. The plates are incubated overnight in a CO₂ incubator (5% CO₂ at 37° C.). Immediately before the assay, the medium is removed and replaced by 200 μl of PBS containing 10 mM LiCl, and the cells are equilibrated with the new medium for 20 minutes. During this interval, cells also are equilibrated with antagonist, added as a 10 μl aliquot of a 20-fold concentrated solution in PBS.

The ³H-inositol phosphate accumulation from inositol phospholipid metabolism is started by adding 10 μl of a solution containing a test compound. To the first well 10 μl are added to measure basal accumulation. Eleven different concentrations of test compound are assayed in the following 11 wells of each plate row. All assays are performed in duplicate by repeating the same additions in two consecutive plate rows.

The plates are incubated in a CO₂ incubator for one hour. The reaction is terminated by adding 15 μl of 50% v/v trichloroacetic acid (TCA), followed by a 40 minute incubation at 4° C. After neutralizing TCA with 40 μl of 1 M Tris, the content of the wells is transferred to a Multiscreen HV filter plate (Millipore) containing Dowex AG1-X8 (200-400 mesh, formate form). The filter plates are prepared by adding 200 μl of Dowex AG1-X8 suspension (50% v/v, water:resin) to each well. The filter plates are placed on a vacuum manifold to wash or elute the resin bed. Each well is washed 2 times with 200 μl of water, followed by 2×200 μl of 5 mM sodium tetraborate/60 mM ammonium formate.

The ³H—IPs are eluted into empty 96-well plates with 200 μl of 1.2 M ammonium formate/0.1 formic acid. The content of the wells is added to 3 ml of scintillation cocktail, and radioactivity is determined by liquid scintillation counting.

### EXAMPLE 7

Receptor Binding Methods

Standard Binding Assays. Binding assays are carried out in a binding buffer containing 50 mM HEPES, pH 7.4, 0.5% BSA, and 5 mM MgCl₂. The standard assay for radioligand (e.g., ¹²⁵I-test compound) binding to membrane fragments comprising P2Y15 GPCR polypeptides is carried out as follows in 96 well microtiter plates (e.g., Dynatech Immulon II Removawell plates). Radioligand is diluted in binding buffer+PMSF/Baci to the desired cpm per 50 μl, then 50 μl aliquots are added to the wells. For non-specific binding samples, 5 μl of 40 μM cold ligand also is added per well. Binding is initiated by adding 150 μl per well of membrane diluted to the desired concentration (10-30 μg membrane protein/well) in binding buffer+PMSF/Baci. Plates are then covered with Linbro mylar plate sealers (Flow Labs) and placed on a Dynatech Microshaker II. Binding is allowed to proceed at room temperature for 1-2 hours and is stopped by centrifuging the plate for 15 minutes at 2,000×g. The supernatants are decanted, and the membrane pellets are washed once by addition of 200 μl of ice cold binding buffer, brief shaking, and recentrifugation. The individual wells are placed in 12×75 mm tubes and counted in an LKB Gammamaster counter (78% efficiency). Specific binding by this method is identical to that measured when free ligand is removed by rapid (3-5 seconds) filtration and washing on polyethyleneiminrie-coated glass fiber filters.

Three variations of the standard binding assay are also used.
1. Competitive radioligand binding assays with a concentration range of cold ligand vs. ¹²⁵I-labeled ligand are carried out as described above with one modification. All dilutions of ligands being assayed are made in 40× PMSF/Baci to a concentration 40× the final concentration in the assay. Samples of peptide (5 μl each) are then added per microtiter well. Membranes and radioligand are diluted in binding buffer without protease inhibitors. Radioligand is added and mixed with cold ligand, and then binding is initiated by addition of membranes.
2. Chemical cross-linking of radioligand with receptor is done after a binding step identical to the standard assay. However, the wash step is done with binding buffer minus BSA to reduce the possibility of non-specific cross-linking of radioligand with BSA. The cross-lining step is carried out as described below.
3. Larger scale binding assays to obtain membrane pellets for studies on solubilization of receptor:ligand complex and for receptor purification are also carried out. These are identical to the standard assays except that (a) binding is carried out in polypropylene tubes in volumes from 1-250 ml, (b) concentration of membrane protein is always 0.5 mg/ml, and (c) for receptor purification, BSA concentration in the binding buffer is reduced to 0.25%, and the wash step is done with binding buffer without BSA, which reduces BSA contamination of the purified receptor.

### EXAMPLE 8

Chemical Cross-Linking of Radioligand to Receptor

After a radioligand binding step as described above, membrane pellets are resuspended in 200 μl per microtiter plate well of ice-cold binding buffer without BSA. Then 5 μl per well of 4 mM N-5-azido-2-nitrobenzoyloxysuccinimide (ANB-NOS, Pierce) in DMSO is added and mixed. The samples are held on ice and UV-irradiated for 10 minutes with a Mineralight R-52G lamp (UVP Inc., San Gabriel, Calif.) at a distance of 5-10 cm. Then the samples are transferred to Eppendorf microfuge tubes, the membranes pelleted by centrifugation, supernatants removed, and membranes solubilized in Laemmli SDS sample buffer for poly-acrylamide gel electrophoresis (PAGE). PAGE is carried out as described below. Radiolabeled proteins are visualized by autoradiography of the dried gels with Kodak XAR film and DuPont image intensifier screens.

### EXAMPLE 9

Membrane Solubilization

Membrane solubilization is carried out in buffer containing 25 mM Tris, pH 8, 10% glycerol (w/v) and 0.2 mM CaCl₂ (solubilization buffer). The highly soluble detergents including Triton X-100, deoxycholate, deoxycholate:lysolecithin, CHAPS, and zwittergent are made up in solubilization buffer at 10% concentrations and stored as frozen aliquots. Lysolecithin is made up fresh because of insolubility upon freeze-thawing and digitonin is made fresh at lower concentrations due to its more limited solubility.

To solubilize membranes, washed pellets after the binding step are resuspended free of visible particles by pipetting and vortexing in solubilization buffer at 100,000×g for 30 minutes. The supernatants are removed and held on ice and the pellets are discarded.

### EXAMPLE 10

Assay of Solubilized Receptors

After binding of ¹²⁵I ligands and solubilization of the membranes with detergent, the intact R:L complex can be assayed by four different methods. All are carried out on ice or in a cold room at 4-10° C.).

Column chromatography (Knuhtsen et al., Biochem. J. 254, 641-647, 1988). Sephadex G-50 columns (8×250 mm) are equilibrated with solubilization buffer containing detergent at the concentration used to solubilize membranes and 1 mg/ml bovine serum albumin. Samples of solubilized membranes (0.2-0.5 ml) are applied to the columns and eluted at a flow rate of about 0.7 ml/minute. Samples (0.18 ml) are collected. Radioactivity is determined in a gamma counter. Void volumes of the columns are determined by the elution volume of blue dextran. Radioactivity eluting in the void volume is considered bound to protein. Radioactivity eluting later, at the same volume as free ¹²⁵I ligands, is considered non-bound.

Polyethyleneglycol precipitation (Cuatrecasas, Proc. Natl. Acad. Sci. USA 69, 318-322, 1972). For a 100 μl sample of solubilized membranes in a 12×75 mm polypropylene tube, 0.5 ml of 1% (w/v) bovine gamma globulin (Sigma) in 0.1 M sodium phosphate buffer is added, followed by 0.5 ml of 25% (w/v) polyethyleneglycol (Sigma) and mixing. The mixture is held on ice for 15 minutes. Then 3 ml of 0.1 M sodium phosphate, pH 7.4, is added per sample. The samples are rapidly (1-3 seconds) filtered over Whatman GF/B glass fiber filters and washed with 4 ml of the phosphate buffer. PEG-precipitated receptor: ¹²⁵I-ligand complex is determined by gamma counting of the filters.

GFB/PEI filter binding (Bruns et al., Analytical Biochem. 132, 74-81, 1983). Whatman GF/B glass fiber filters are soaked in 0.3% polyethyleneimine (PEI, Sigma) for 3 hours. Samples of solubilized membranes (25-100 μl) are replaced in 12×75 mm polypropylene tubes. Then 4 ml of solubilization buffer without detergent is added per sample and the samples are immediately filtered through the GFB/PEI filters (1-3 seconds) and washed with 4 ml of solubilization buffer. CPM of receptor: ¹²⁵I-ligand complex adsorbed to filters are determined by gamma counting.

Charcoal/Dextran (Paul and Said, Peptides 7[Suppl. 1], 147-149, 1986). Dextran T70 (0.5 g, Pharmacia) is dissolved in 1 liter of water, then 5 g of activated charcoal (Norit A, alkaline; Fisher Scientific) is added. The suspension is stirred for 10 minutes at room temperature and then stored at 4° C. until use. To measure R:L complex, 4 parts by volume of charcoal/dextran suspension are added to 1 part by volume of solubilized membrane. The samples are mixed and held on ice for 2 minutes and then centrifuged for 2 minutes at 11,000×g in a Beckman microfuge. Free radioligand is adsorbed charcoal/dextran and is discarded with the pellet. Receptor: ¹²⁵I-ligand complexes remain in the supernatant and are determined by gamma counting.

### EXAMPLE 11

Receptor Purification

Binding of biotinyl-receptor to GH₄ Cl membranes is carried out as described above. Incubations are for 1 hour at room temperature. In the standard purification protocol, the binding incubations contain 10 nM Bio-S29. ¹²⁵I ligand is added as a tracer at levels of 5,000-100,000 cpm per mg of membrane protein. Control incubations contain 10 μM cold ligand to saturate the receptor with non-biotinylated ligand.

Solubilization of receptor:ligand complex also is carried out as described above, with 0.15% deoxycholate:lysolecithin in solubilization buffer containing 0.2 mM MgCl₂, to obtain 100,000×g supernatants containing solubilized R:L complex.

Immobilized streptavidin (streptavidin cross-linked to 6% beaded agarose, Pierce Chemical Co.; “SA-agarose”) is washed in solubilization buffer and added to the solubilized membranes as 1/30 of the final volume. This mixture is incubated with constant stirring by end-over-end rotation for 4-5 hours at 4-10° C. Then the mixture is applied to a column and the non-bound material is washed through. Binding of radioligand to SA-agarose is determined by comparing cpm in the 100,000×g supernatant with that in the column effluent after adsorption to SA-agarose. Finally, the column is washed with 12-15 column volumes of solubilization buffer+0.15% deoxycholate:lysolecithin+ 1/500 (vol/vol) 100×4 pase.

The streptavidin column is eluted with solubilization buffer+0.1 mM EDTA+0.1 mM EGTA+0.1 mM GTP-gamma-S (Sigma)+0.15% (wt/vol) deoxycholate:lysolecithin+ 1/1000 (vol/vol) 100.times.4pase. First, one column volume of elution buffer is passed through the column and flow is stopped for 20-30 minutes. Then 3-4 more column volumes of elution buffer are passed through. All the eluates are pooled.

Eluates from the streptavidin column are incubated overnight (12-15 hours) with immobilized wheat germ agglutinin (WGA agarose, Vector Labs) to adsorb the receptor via interaction of covalently bound carbohydrate with the WGA lectin. The ratio (vol/vol) of WGA-agarose to streptavidin column eluate is generally 1:400. A range from 1:1000 to 1:200 also can be used. After the binding step, the resin is pelleted by centrifugation, the supernatant is removed and saved, and the resin is washed 3 times (about 2 minutes each) in buffer containing 50 mM HEPES, pH 8, 5 mM MgCl₂, and 0.15% deoxycholate:lysolecithin. To elute the WGA-bound receptor, the resin is extracted three times by repeated mixing (vortex mixer on low speed) over a 15-30 minute period on ice, with 3 resin columns each time, of 10 mM N—N′—N″-triacetylchitotriose in the same HEPES buffer used to wash the resin. After each elution step, the resin is centrifuged down and the supernatant is carefully removed, free of WGA-agarose pellets. The three, pooled eluates contain the final, purified receptor. The material non-bound to WGA contain G protein subunits specifically eluted from the streptavidin column, as well as non-specific contaminants. All these fractions are stored frozen at −90° C.

### EXAMPLE 12

Identification of Test Compounds that Bind to P2Y15 GPCR Polypeptides

Purified P2Y15 GPCR polypeptides comprising a glutathione-S-transferase protein and absorbed onto glutathione-derivatized wells of 96-well microtiter plates are contacted with test compounds from a small molecule library at pH 7.0 in a physiological buffer solution. P2Y15 GPCR polypeptides comprise an amino acid sequence shown in SEQ ID NO:2. The test compounds comprise a fluorescent tag. The samples are incubated for 5 minutes to one hour. Control samples are incubated in the absence of a test compound.

The buffer solution containing the test compounds is washed from the wells. Binding of a test compound to a P2Y15 GPCR polypeptide is detected by fluorescence measurements of the contents of the wells. A test compound which increases the fluorescence in a well by at least 15% relative to fluorescence of a well in which a test compound was not incubated is identified as a compound which binds to a P2Y15 GPCR polypeptide.

### EXAMPLE 13

Identification of a Test Compound which Decreases P2Y15 GPCR Gene Expression

A test compound is administered to a culture of human gastric cells and incubated at 37° C. for 10 to 45 minutes. A culture of the same type of cells incubated for the same time without the test compound provides a negative control.

RNA is isolated from the two cultures as described in Chirgwin et al., Biochem. 18, 5294-99, 1979). Northern blots are prepared using 20 to 30 μg total RNA and hybridized with a ³²P-labeled P2Y15 GPCR-specific probe at 65° C. in Express-hyb (CLONTECH). The probe comprises at least 11 contiguous nucleotides selected from the complement of SEQ ID NO:1. A test compound which decreases the P2Y 15 GPCR-specific signal relative to the signal obtained in the absence of the test compound is identified as an inhibitor of P2Y15 GPCR gene expression.

### EXAMPLE 14

Treatment of a Disease in which Human P2Y15 GPCR is Overexpressed with a Reagent which Specifically Binds to a P2Y15 GPCR Gene Product

Synthesis of antisense P2Y15 GPCR oligonucleotides comprising at least 11 contiguous nucleotides selected from the complement of SEQ ID NO:1 is performed on a Pharmacia Gene Assembler series synthesizer using the phosphoramidite procedure (Uhlmann et al., Chem. Rev. 90, 534-83, 1990). Following assembly and deprotection, oligonucleotides are ethanol-precipitated twice, dried, and suspended in phosphate-buffered saline (PBS) at the desired concentration. Purity of these oligonucleotides is tested by capillary gel electrophoreses and ion exchange HPLC. Endotoxin levels in the oligonucleotide preparation are determined using the Luminous Amebocyte Assay (Bang, Biol. Bull. (Woods Hole, Mass.) 105, 361-362, 1953).

The antisense oligonucleotides are administered to a patient. The severity of the patient's disease is decreased.

### EXAMPLE 15

Tissue-Specific Expression of P2Y15 GPCR

In an analysis of P2Y15 GPCR gene expression, 25 μg of total RNA from the following sources were used as template in reactions to synthesize first-strand cDNA for expression profiling: Human Total RNA Panel I-V (Clontech Laboratories, Palo Alto, Calif., USA), normal human lung primary cell lines (BioWhittaker Clonetics, Walkersville, Md., USA), human umbilical vein endothelial cells (HUVECs) (Kurabo, Osaka, Japan), several common cell lines (ATCC, Washington, D.C.), and various cells purified from peripheral blood. First-strand cDNA was synthesized using oligo (dT) Nippon Gene Research Laboratories, Sendai, Japan) and the SUPERSCRIPT™ First-Strand Synthesis System for RT-PCR (Life Technologies, Rockville, Md.) according to the manufacturer's protocol. For these samples, 1/1250^{th} of the synthesized first-strand cDNA was subsequently used as template for quantitative PCR. Additional samples were purchased as presynthesized cDNAs (Human Immune System MTC Panel and Human Blood Fractions MTC Panel, Clontech Laboratories), and for these, 10 ng of cDNA was used as template for quantitative PCR.

Quantitative PCR was performed in a LightCycler (Roche Molecular Biochemicals, Indianapolis, Ind.) with oligonucleotide primers 5′-TTCGGATCGAATCTCGCCTGCT-3′ (SEQ ID NO:7) and 5′-TGCTTGCTCAAGGTTCCCGCTTA-3′ (SEQ ID NO:8) in the presence of the DNA-binding fluorescent dye SYBR Green I. Results were then converted into copy numbers of the gene transcript per ng of template cDNA by fitting to a standard curve. The standard curve was derived by simultaneously performing the quantitative PCR reaction on PCR products of known concentrations amplified beforehand from the target gene.

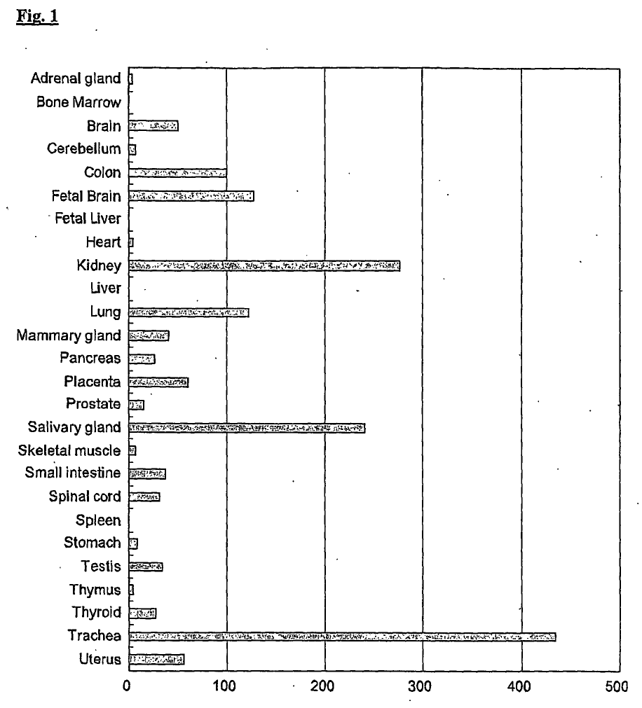

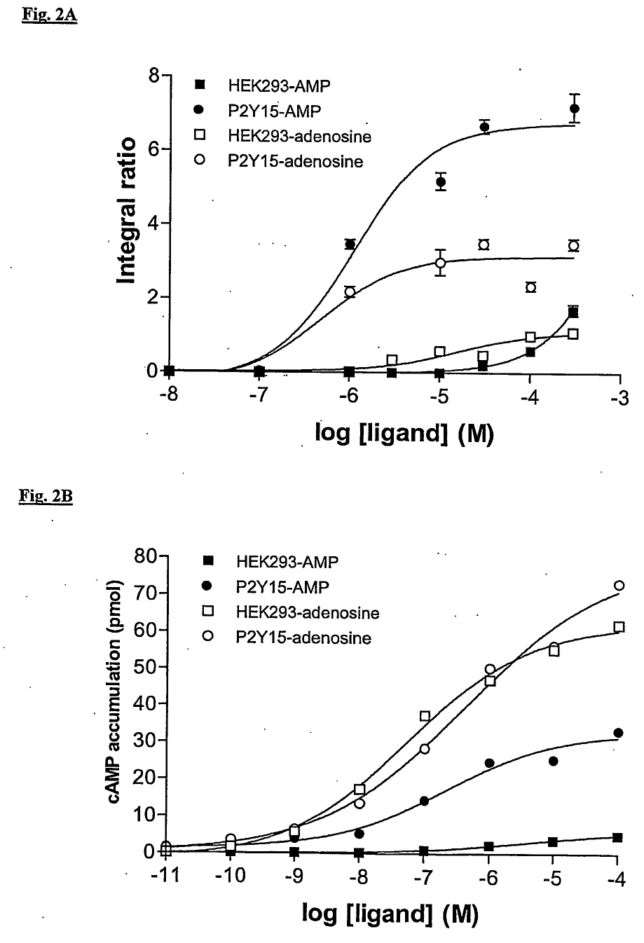

To correct for differences in mRNA transcription levels per cell in the various tissue types, a normalization procedure was performed using similarly calculated expression levels of five different housekeeping genes: glyceraldehyde-3-phosphatase (G3PDH), hypoxanthine guanine phophoribosyl transferase (HPRT), beta-actin, porphobilinogen deaminase (PBGD), and beta-2-microglobulin. The level of house-keeping gene expression is considered to be relatively constant for all tissues (Adams et al., 1993, Adams et al., 1995, Liew et al., 1994) and therefore can be used as a gauge to approximate relative numbers of cells per ng of cDNA template. Expression levels of the five housekeeping genes in all tissue samples were measured in three independent reactions per gene using the LightCycler and a constant amount (25 μg) of starting RNA. The calculated copy numbers for each gene, derived from comparison with simultaneously reacted standards of known concentrations, were recorded and converted into a percentage of the sum of the copy numbers of the gene in all tissue samples. For each tissue sample, the sum of the percentage values for each gene was calculated, and a normalization factor was calculated by dividing the sum percentage value for each tissue by the sum percentage value of one of the tissues arbitrarily selected as a standard. To normalize an experimentally obtained value for the expression of a particular gene in a tissue sample, the obtained value was multiplied by the normalization factor for the tissue tested. This normalization method was used for all tissues except those derived from the Human Blood Fractions MTC Panel, which were normalized against the single housekeeping gene, beta-2-microglobulin, due to wide variation in other housekeeping gene expression in these tissues depending on activation status. The results of this expression profiling are given in FIG. 1, showing the normalized values for the copy numbers of mRNA per 10 ng of first-strand cDNA in each sample tested.

### EXAMPLE 16

### Determination of the P2Y15 GPCR Ligand

The polynucleotide of SEQ ID NO: 1 was cloned using human genomic DNA as a template and performing PCR with primers 5′-GCCAAACTGAACTCTCTTGTTTTCTTGC-3′ (SEQ ID NO:9) and 5′-GCCCTGGCTTTGGCACATGATTAC-3′ (SEQ ID NO:10) and a blend of HotStarTaq (Qiagen, Hilden, Germany) and Pfu Turbo (Stratagene, La Jolla, Calif.) polymerases. The PCR products were cloned into pCRII-TOPO (Invitrogen, Carlsbad, Calif.), cycle-sequenced with an ABI Prism Dye Terminator Cycle Sequencing Reaction Kit (Applied Biosystems, Foster City, Calif.), and analyzed on an ABI Prism 377 sequencing system (Applied Biosystems). The cDNA was then subcloned into a modified pDisplay vector (Invitrogen) to append an N-terminal HA epitope and Ig signal sequence. The expression vector pDisplay-P2Y15 GPCR construct obtained was transfected into human embryonic kidney 293 cells using Lipofectamine (Invitrogen). Expression on the cell surface was verified by staining the cells with phycoerythrin-labeled anti-HA antibody (Santa Cruz Biotechnology, Santa Cruz, Calif.) and measuring cell-bound fluorescence on a FACSort (Becton-Dickinson, Franklin Lakes, N.J.). Stably transfected clones were then generated by limiting dilution followed by selection in G418. Finally, cell-surface expression of the P2Y15 GPCR polypeptide in the clones was reconfirmed by FACS.

Ligand screening was performed in a Ca²⁺ flux assay as follows. Stably transfected P2Y15 GPCR-expressing cells were seeded into 96-well plates and incubated overnight at 37° C. in a tissue culture incubator. The culture medium was aspirated and replaced with 100 l of loading buffer containing 0.1% BSA, 20 mM HEPES, 1 mM probenecid, 0.01% pluronic F127, and 1 M Fluo-3-AM (Molecular Probes, Eugene, Oreg.) in HBSS, and incubated for 1 hour at room temperature. The cells were then washed gently 3 times with wash buffer containing 0.1% BSA, 20 mM HEPES, 1 mM probenecid in HBSS. The washed cells were placed in an FDSS6000 functional drug screening system (Hamamatsu Photonics, Hamamatsu, Japan) and changes in cellular fluorescence were measured after adding serial dilutions of potential ligands. A panel of about 130 potential ligands was assembled by selecting known ligands of the GPCRs most closely related to P2Y15 GPCR as well as several naturally occurring chemical relatives of the ligands. The library included various bioactive lipids, eicosanoids, peptides, cannabinoids, chemokines, nucleosides, nucleotides and chemically related substances. The ligands were genereally purchased from either Sigma or R&D Systems.

Among the potential ligands tested, only AMP and adenosine were able to induce a response in the transfectants while not inducing a similar response in either nontransfected HEK293 cells or HEK293 cells stably transfected with the control orphan GPCR P2Y8 in an identical vector construct. We detected a calcium response with an EC50 of 920 nM for AMP and 670 nM for adenosine (FIG. 2A). Both stable transfectants and nontransfected cells mobilized calcium in response to ATP, AMP OR ADENOSINE RECEPTOR LIGAND, and UTP, consistent with previous reports of HEK293 endogenously expressing P2Y1 and P2Y2 receptors (Schachter, J. B., Sromek, S. M., Nicholas, R. A., and Harden, T. K. (1997) Neuropharmacology 36, 1181-1187). Further analysis by RT-PCR showed that the nucleotide receptors P2Y4, P2Y12, and P2Y13 and the adenosine receptors A2A and A2B (previously reported in (Sunahara, R. K., Dessauer, C. W., and Gilman, A. G. (1996) Annu Rev Pharmacol Toxicol 36, 461-480)) are also expressed in HEK293 cells. Despite the endogenous expression of the adenosine receptors, calcium mobilization responses to adenosine in nontransfected cells could only be detected at very high adenosine concentrations, and showed only a very weak response.

### EXAMPLE 17

Cyclic AMP Production Assay

To determine the effect of P2Y15 stimulation on adenylyl cyclase activity, cyclic AMP accumulation in P2Y15-EK293 stable transfectants in response to AMP and adenosine was measured with the Tropix cAMP screen (Applied Biosystems) according to the manufacturer's protocol. Briefly, stable transfectants and control cells (1×10⁵ cells/well) were cultured for two hours with or without 1 μM pertussis toxin, then treated for 30 min with 10 μM forskolin and serial dilutions of AMP or adenosine. The cells were then lysed and the cAMP produced was measured by a cAMP-specific ELISA. Concentrations of cAMP produced were calculated by comparing against cAMP standards measured simultaneously. Stimulation with either ligand alone gave only minor responses barely above the detection limit. In the presence of 10 μM forskolin, however, both AMP and adenosine induced the generation of cyclic AMP in a dose dependent manner, with an EC₅₀ of 214 nM for AMP and 327 nM for adenosine (FIG. 2B). Nontransfected HEK293 cells similarly generated cyclic AMP in response to adenosine, likely due to the stimulation of endogenously expressed adenosine receptors, but did not respond strongly to AMP. The production of cyclic AMP in response to either ligand was not affected by pretreatment of the cells for two hours with 1 μM pertussis toxin, indicating that P2Y15 is coupled to an adenylate cyclase-stimulating G protein.

### EXAMPLE 18

Receptor Binding Assay

10⁵ cells per well in 96-well plates were washed twice for 1 h with DMEM medium. Then wheatgerm agglutinin SPA beads (Amersham) at 1 mg/well were added, followed 1 h later by increasing concentrations of [³H]-Adenosine or [3H]-AMP (Amersham), in a constant volume of HBS: 10 mM Hepes, 130 mM NaCl, 5 mM KCl, 1 mM MgCl₂, 1 mM CaCl₂ and 1 g/l glucose (6 μl/well)). After incubating at 4° C. for 16 h, the plates were centrifuged for 10 min at 1500 rpm and then scintillation measured on a TopCount automated scintillation counter. Binding of [³²P]-AMP was carried out under the same conditions except that instead of using SPA beads, at the end of the incubation, cells were washed three times by vacuum filtration and 100 μl scintillation fluid was added to the wells. Non-specific binding measurements were carried out under the same conditions but with either an excess of cold ligand (2.5 mM. K_{d} values were determined by non-linear regression using the program Prism (Graph Pad).

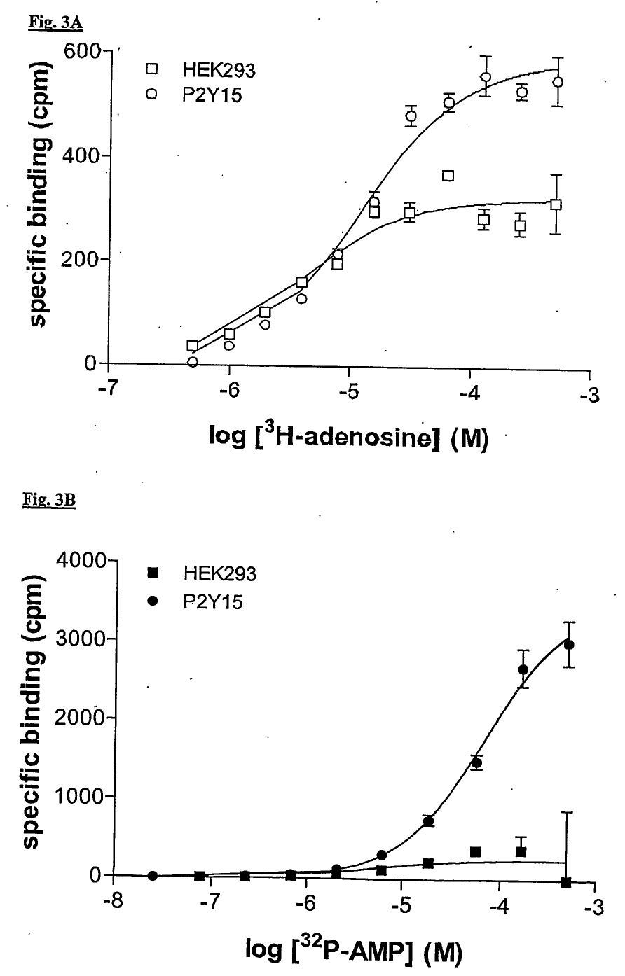

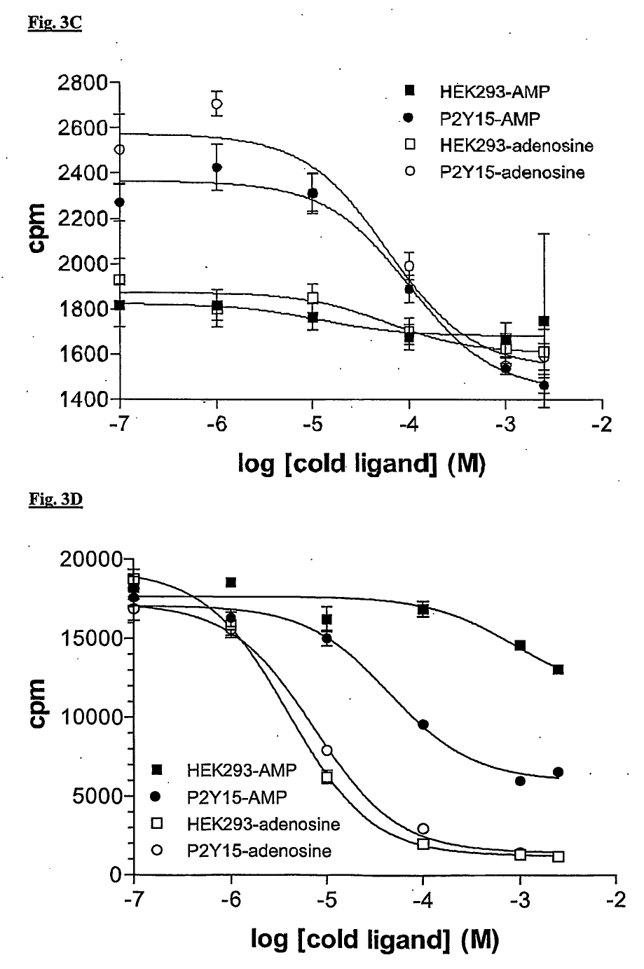

Saturation binding analysis of the ligands to the P2Y15 receptor in stable transfectants gave K_{d} values of 12.0 μM for ³H-adenosine (FIG. 3A) and 18.6 μM for ³H-AMP (data not shown). Since AMP can be dephosphorylated to adenosine by ectonucleotidases, we repeated the binding analysis with adenosine 5′-[³²P] mono-phosphate to confirm that the binding being measured was AMP and not adenosine. This resulted in a similar binding curve with a K_{d} of 18.8 μM (FIG. 3B), indicating that AMP itself, and not a breakdown product, was binding to the receptor.

### EXAMPLE 19

Competitive Binding Assay

Competitive binding experiments were carried out as follows: 10⁵ cells per well in 96-well plates were washed twice for 1 h with DMEM medium. Then wheatgerm agglutinin SPA beads (Amersham) at 1 mg/well were added, followed 1 h later by 10 μM [³H]-Adenosine or 10 μM [³H]-AMP (Amersham), and increasing concentrations of unlabeled ligand in a constant volume of HBS: 10 mM Hepes, 130 mM NaCl, 5 mM KCl, 1 mM MgCl₂, 1 mM CaCl₂ and 1 g/l glucose (6 μl/well)). After incubating at 4° C. for 16 h, the plates were centrifuged for 10 min at 1500 rpm and then scintillation measured on a TopCount automated scintillation counter. Kᵢ values were determined by non-linear regression using the program Prism (Graph Pad).

Unlabled adenosine was able to block the binding of 10 μM ³H-AMP to transfectants (Kᵢ=39.8 μM) with a potency similar to that of unlabled AMP (FIG. 3C). On the other hand, while unlabeled AMP was able to block a large proportion of the binding of 10 μM ³H-adenosine to transfectants (Kᵢ=24.6 μM), it could not block the binding as completely as unlabled adenosine and had little effect in blocking ³H-adenosine binding to nontransfected HEK293 cells (FIG. 3D). While these results give support to the idea that both AMP and adenosine bind to P2Y15, because neither AMP nor adenosine can antagonize the binding of ³H-AMP to nontransfected HEK293 cells, the results also demonstrate a lack of specific AMP binding sites on the nontransfected cells.

### EXAMPLE 20

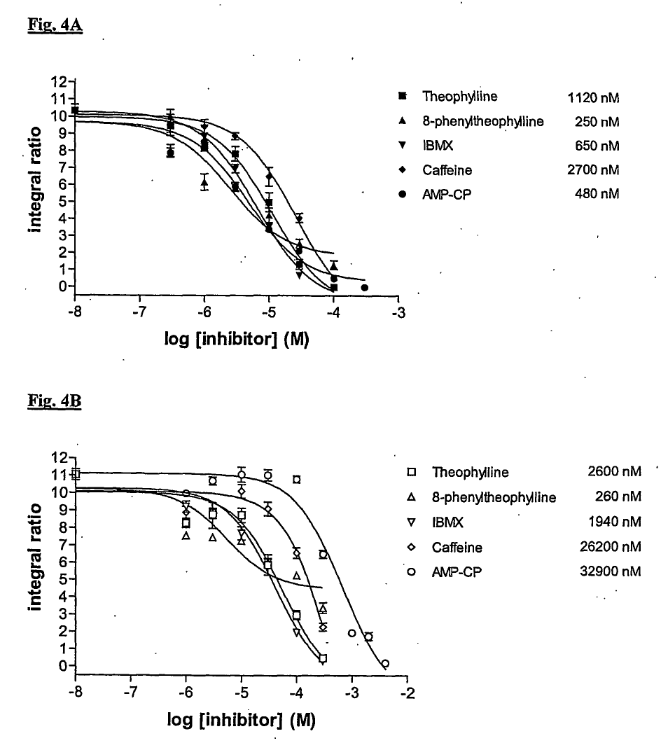

To test whether any of the known non-specific antagonists of adenosine receptors could antagonize P2Y15, calcium mobilization assays was performed in the presence of varying concentrations of theophylline, 8-phenyltheophylline, 3-isobutyl-1 methylxanthine (IBMX), and caffeine. All of these compounds were able to block the calcium mobilization induced by AMP and adenosine, with Kᵢ values for blocking AMP ranging from 250 nM for 8-phenyltheophylline to 2700 nM for caffeine and Kᵢ values for blocking adenosine ranging from 260 nM for 8-phenyltheophylline to 26200 nM for caffeine. (FIGS. 4A and B).

### EXAMPLE 21

Adenosine 5′-(α,β-methylene)diphosphate (AMP-CP), which as a potent inhibitor of ectonucleotidases has been used in the past to provide evidence that blocking the conversion of AMP to adenosine can effectively block cellular responses to AMP, was effective in blocking calcium responses to not only AMP but also to adenosine, indicating that this compound can also directly inhibit binding to the P2Y15 receptor (FIG. 4).

### EXAMPLE 22

Mast Cell Expression of P2Y15 Detected by Microarray Analysis

Target Preparation

Total RNA was prepared from human umbilical cord blood-derived mast cells (HCMC) according to the method for generating HCMC in vitro established by H. Saito (H. Saito et al. J. Immunol. 157:343-350, 1996). The HCMC so generated appear to resemble human lung mast cells in terms of their intracellular protease profiles, their histamine release characteristics, and their pharmacological properties (H. Saito et al. J. Immunol. 157:343-350;1996, Y. Igarashi et al. Clin. Exp. Allergy 26:597-602;1996, and H. Nagai et al. Clin. Exp. Allergy 28:1228-1236;1998). The HCMC, therefore, are useful for biological and pharmacological studies of lung mast cells and for the development of new anti-asthma drugs. Total RNA from the HCMC was isolated using Trizol™ (Invitrogen Corp., Carlsbad, Calif., USA) according to the manufacturer's protocol. Five micrograms of the total RNA was then added to a reaction mix in a final volume of 12 μl, containing bacterial control mRNAs (2.5 pg/μl araB/entF, 8.33 pg/μl fixB/gnd and 25 pg/μl hisB/leuB) and 1.0 μl of 0.5 pmol/μl T7-(dT)₂₄ oligonucleotide primer. The mixture was incubated for 10 min at 70° C. and chilled on ice. With the mixture remaining on ice, 4 μl of 5× first-strand buffer, 2 μl 0.1 M DTT, 1 μl of 10 mM dNTP mix and 1 μl Superscript™ II RNase H-reverse transcriptase (200 U/μl) was added to make a final volume of 20 μl, and the mixture incubated for 1 h in a 42° C. water bath. Second-strand cDNA was synthesized in a final volume of 150 μl, in a mixture containing 30 μl of 5× second-strand buffer, 3 μl of 10 mM dNTP mix, 4 μl of Escherichia coli DNA polymerase I (10 U/μl) and 1 μl of kNase H (2 U/μl) for 2 h at 16° C. The cDNA was purified using a Qiagen QIAquick purification kit, dried down, and resuspended in IVT reaction mix, containing 3.0 μl nuclease-free water, 4.0 μl 10× reaction buffer, 4.0 μl 75 mM ATP, 4.0 μl 75 mM GTP, 3.0 μl 75 mM CTP, 3.0 μl 75 mM UTP, 7.5 μl 10 mM Biotin 11-CTP, 7.5 μl 10 mM Biotin 11-UTP (PerkinElmer Life Sciences Inc. Boston, Mass., USA) and 4.0 μl enzyme mix. The reaction mix was incubated for 14 h at 37° C. and cRNA target purified using an RNeasy® kit (Qiagen). cRNA yield was quantified by measuring the UV absorbance at 260 nm, and fragmented in 40 mM Tris-acetate (TrisOAc) pH 7.9, 100 mM KOAc and 31.5 mM MgOAc, at 94° C. for 20 min. This results typically in a fragmented target with a size range between 100 and 200 bases.

Array Hybridization

Ten micrograms of fragmented target cRNA per array was used for hybridization to UniSet Human I and II Bioarrays (AmershamBiosciences) in 260 μl of hybridization solution containing 78 μl Amersham Hyb buffer component A and 130 μl Amersham Hyb buffer component B. The hybridization solution was heated at 90° C. for 5 min to denature the cRNA and chilled on ice. The sample was vortexed for 5 s at maximum speed, and 250 μl injected into the inlet port of the hybridization chamber. The slides were loaded into a ISF-4-W shaking incubator (Kuhner, Birsfelden, Switzerland), with the hybridization chambers facing up. Slides were incubated for 24 h at 37° C., while shaking at 300 r.p.m.

Post-Hybridization Processing Using Streptavidin-Cy5

The slides were removed from the ISF-4-W shaker, and the hybridization chamber removed from each slide. Each slide was briefly rinsed in TNT buffer (0.1 M Tris-HCl pH 7.6, 0.15 M NaCl, 0.05% Tween-20) at room temperature, and then washed in TNT buffer at 42° C. for 60 min. The signal was developed using a 1:500 dilution of streptavidin-Cy5 (AmershamBiosciences), for 30 min at room temperature. Excess dye was removed by washing four times with TNT buffer, for 5 min each, at room temperature. Slides were rinsed in 0.05% Tween-20 and dried under nitrogen gas. Processed slides were scanned using an Axon GenePix 4000B Scanner with the laser set to 635 nm, the photomultiplier tube (PMT) voltage to 600 and the scan resolution to 10 μm. Images were acquired with the Axon GenePixPro v4.0 Scanning Software (AmershamBiosciences), and analyzed using the CodeLink™ Expression Analysis Software (AmershamBiosciences).

Data Analysis

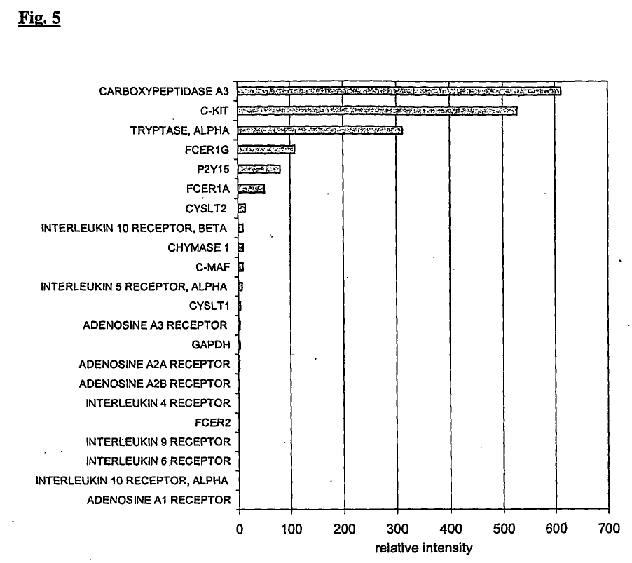

CodeLink™ Expression Analysis Software (AmershamBiosciences) automatically creates signal data for each spotted dot as a Microsoft Excel formatted spreadsheet. The data was analyzed using the computer program Spotfire Decision Site 7.0 (Spotfire Japan K.K., Tokyo, Japan) to determine the relative intensity of expression for each of the approximately 20,000 genes represented on the two arrays. As a result of this analysis, the P2Y15 gene transcript was found to be consistently expressed higher among the top approximately 1% of genes in terms of relative intensity. FIG. 5 shows the expression intensity of P2Y15 in relation to other well known genes expressed in mast cells.

### REFERENCES

1. Nandanan E, Jang S Y, Moro S, Kim H O, Siddiqui M A, Russ P, Marquez V E, Busson R, Herdewijn
2. P, Harden T K, Boyer J L, Jacobson K A. Synthesis, biological activity, and molecular modeling of ribose-modified deoxyadenosine bisphosphate analogues as P2Y(1) receptor ligands. J Med. Chem. 2000 Mar. 9;43(5):829-42.
3. Major D T, Halbfinger E, Fischer B. Molecular recognition of modified adenine nucleotides by the P2Y(1)-receptor. 2. A computational approach. J Med Chem. 1999 Dec. 30;42(26):5338-47.
3. Halbfinger E, Major D T, Ritzmann M, Ubl J, Reiser G, Boyer J L, Harden K T, Fischer B. Molecular recognition of modified adenine nucleotides by the P2Y(1)-receptor. 1. A synthetic, biochemical, and NMR approach. J Med Chem. 1999 Dec. 30;42(26):5325-37.
4. Moro S, Hoffmann C, Jacobson K A. Role of the extracellular loops of G protein-coupled receptors in ligand recognition: a molecular modeling study of the human P2Y1 receptor. Biochemistry. 1999 Mar. 23;38(12):3498-507.
5. Moro S, Guo D, Camaioni E, Boyer J L, Harden T K, Jacobson K A. Human P2Y1 receptor: molecular modeling and site-directed mutagenesis as tools to identify agonist and antagonist recognition sites. J Med Chem. 1998 Apr. 23;41(9): 1456-66.
6. Webb T E, Simon J, Barnard E A. Regional distribution of [35S]2′-deoxy 5′-O-(1-thio) ATP binding sites and the P2Y1 messenger RNA within the chick brain. Neuroscience. 1998 June;84(3):825-37.
7. Homolya L, Watt W C, Lazarowski E R, Koller B H, Boucher R C. Nucleotide-regulated calcium signaling in lung fibroblasts and epithelial cells from normal and P2Y(2) receptor (−/−) mice. J Biol. Chem. 1999 Sep. 10;274(37):26454-60.
8. Leon C, Hechler B, Vial C, Leray C, Cazenave J P, Gachet C. The P2Y1 receptor is an AMP OR ADENOSINE RECEPTOR LIGAND receptor antagonized by ATP and expressed in platelets and megakaryoblastic cells. FEBS Lett. 1997 Feb. 10;403(1):26-30.
9. Dehaye J P, Moran A, Marino A. Purines, a new class of agonists in salivary glands? Arch Oral Biol. 1999 May;44 Suppl 1:S39-43.
10. King B F, Townsend-Nicholson A, Burnstock G. Metabotropic receptors for ATP and UTP: exploring the correspondence between native and recombinant nucleotide receptors. Trends Pharmacol Sci. 1998 December; 19(12):506-14.
11. Saleh A, Figarella C, Kammouni W, Marchand-Pinatel S, Lazdunski A, Tubul A, Brun P, Merten M D. Pseudomonas aeruginosa quorum-sensing signal molecule N-(3-oxododecanoyl)-L-homoserine lactone inhibits expression of P2Y receptors in cystic fibrosis tracheal gland cells. Infect Immun. 1999 October;67(10):5076-82.
12. Cressman V L, Lazarowski E, Homolya L, Boucher R C, Koller B H, Grubb B R. Effect of loss of P2Y(2) receptor gene expression on nucleotide regulation of murine epithelial Cl(−) transport. J Biol. Chem. 1999 Sep. 10;274(37):26461-8.

1. A method for detecting the activity of P2Y15 in a sample comprising the steps of:
a) incubating a sample comprising P2Y15 and a ligand under conditions which allow binding of P2Y15 and the ligand, and
b) detecting a second messenger, wherein said ligand is AMP or adenosine receptor ligand.

2. The method of claim 1 further comprising the steps of:
a) incubating a second sample comprising P2Y15 in the absence of the ligand under conditions which allow binding of P2Y15 and the ligand, and
b) detecting a second messenger.

3. The method of claim 1 wherein said sample comprises cells expressing P2Y15.

4. The method of claim 1 wherein said sample comprises cell membranes bearing P2Y15.

5. A method of screening for an agent to modulate P2Y15 activity using cells expressing P2Y15, said method comprising:
a) incubating a first sample of said cells in the presence of said agent and a second sample of said cells in the absence of said agent, both said samples under conditions which allow binding of AMP or adenosine receptor ligand to P2Y15;
b) detecting a signalling activity of P2Y15 polypeptide in said first and second samples, and
c) comparing the results of said second messenger assays for said first and second samples.

6. (canceled)

7. (canceled)

8. (canceled)

9. A method of identifying an agent that modulates the function of P2Y15, said method comprising:
a) contacting a P2Y15 polypeptide in the presence and absence of an agent under conditions permitting the binding of said AMP or adenosine receptor ligand to said P2Y15 polypeptide; and
b) measuring the binding of said P2Y15 polypeptide to said agent, relative to the binding in the absence of said agent, wherein an agent which changes binding is identified as a potential therapeutic agent for decreasing or increasing the function of P2Y15.

10. The method of claim 9 wherein said measuring is performed using a method selected from label displacement, surface plasmon resonance, fluorescence resonance energy transfer, fluorescence quenching, and fluorescence polarization.

11. The method of claim 5 wherein said agent is selected from the group consisting of a natural or synthetic peptide, a polypeptide, an anti-body or antigen-binding fragment thereof, a lipid, a carbohydrate, a nucleic acid, and a small organic molecule.

12. The method of claim 5 wherein said step of measuring a signalling activity of said P2Y15 polypeptide comprises detecting a change in the level of a second messenger.

13. The method of claim 5 wherein the step of detecting a signalling activity comprises measurement of guanine nucleotide binding or exchange, adenylate cyclase activity, cAMP, protein kinase C activity, phosphatidylinosotol breakdown, diacylglycerol, inositol triphosphate, intracellular calcium, arachinoid acid concentration, MAP kinase activity, tyrosine kinase activity, reporter gene expression.

14. (canceled)

15. (canceled)

16. (canceled)

17. (canceled)

18. (canceled)
